(19) United States
(12) Patent Application Publication
(10) Pub. No.: US 2003/0147936 A1
(43) Pub. Date: Aug. 7, 2003

(54) Prostatic hormonal implants treatment of prostate cancer

(76) Inventor: Velayudhan Sahadevan, Beckley, WV (US)

Correspondence Address: Dr. V. Sahadevan, Dag Hammarskjold Cancer Treatment Center, 155 Dry Hill Road, Beckley, WV, 25801, US

(21) Appl. No.: 10/072,416

(22) Filed: Feb. 7, 2002

(51) Int. Cl. A61K 31/56

(52) U.S. Cl. 424/426; 514/170

(57) ABSTRACT

An improved method and products for the primary hormonal treatment of early stage, low and intermediate risk prostate cancers by prostatic implants of androgen suppressive drugs formulated as fused with a lipoid carrier or encapsulated in microcapsules or in Silastic capsules is provided. Such prostatic implants renders a constant slow-release of their contents to the prostate for extended periods by biodegradation and diffusion. It facilitates higher prostatic and lower systemic concentrations of androgen suppressive hormones. Because of their high prostatic and lower systemic concentrations, tumor control is much improved and the their systemic toxicity is minimized. Tumor control after such primary hormonal implant treatment is followed by clinical examinations and the biochemical tumor control is followed by periodic estimations of serum levels of PSA and acid phosphatase. More complex and expensive surgery or radiation therapy for this group of good prognostic early stage prostate cancer is reserved for those patients failing to this primary hormonal treatment. It will preserve potency more than by surgery or radiation therapy. Furthermore, it would reduce the cost of treatment for early stage prostate cancer significantly. Androgen suppressive hormonal implants to the prostate before, during or after lower dose conventional radiation therapy would also facilitate equal or better cure rates of localized prostate cancer as compared to the more complex and toxic higher dose radiation therapy.

### BACKGROUND—FIELD OF INVENTION

This invention relates to natural and synthetic chemical hormonal compositions for the treatment of prostate cancer, especially to improved androgen suppressive hormonal treatments by prostatic implants of slow-release androgen suppressive formulations by diffusion and biodegradation, maintaining high concentrations of said formulations in the prostate and maintaining low but sufficient blood levels to effect the hypothalamic-pituitary LHRH, FSH and LH mediated androgen synthesis with minimal systemic toxicity.

### BACKGROUND—DESCRIPTION OF PRIOR ART

Heretofore, hormone treatment of prostate is given by per oral, subcutaneous, intramuscular or intravenous injections. Because of the systemic distribution of such administrated hormones, only a very small amount of hormone reaches the target cancer cells in the prostate. A great percentage of the systemically administered hormone is rapidly metabolized and eliminated from the body and hence it is wasted. Therefore patients have to take larger quantities of these hormones daily. It increases the undesirable side effects of hormone treatment making it unsafe for some patients. Daily systemic administration of the hormones also adds to the cost of these medications and hence unaffordable to some patients. Because of the very low concentration of the systemically administrated hormone reaching the cancer cells, it may not even be adequately effective in some patients.

Both normal and tumor cells of the prostate gland are sensitive to androgen deprivation. Interference with androgen signaling pathways will generate proliferative arrest of both the normal and tumor cells. Furthermore, the androgen deprivation might cause cancer cells to differentiate into a phenotype that is less malignant and to programmed, apoptotic cell death. The proliferative arrest is manifested by a reduction in PSA. Cellular production of PSA is in part controlled by PSA gene, which is regulated by androgen. Interference with androgen regulated PSA gene promotion by androgen deprivation therapy result in decreased PSA production (1; Carroll P. R. et.al, Cancer of the Prostate, In Cancer, Principles and Practice of Oncology, 6^{th} edition, Vol. 1; DeVita, Jr. et al (Ed), 2001 page 1460; (Ref. # 574)) Likewise androgen suppression therapy induced proliferative arrest, that is differentiation to a less malignant phenotypic cancer cells and the apoptotic cell death, all will contribute to decreased cellular synthesis of PSA. Androgen suppression therapy alone is known to induce substantial decrease of the volume of the prostate. Such volume reduction by androgen suppression therapy is routinely used to make bulky prostatic tumors to a smaller size before radioactive seed implant therapy. Volume reduction facilitates an even distribution of radioactive seeds for patients with large prostate gland. The androgen suppression induced programmed cell death and the consequent volume reduction of the prostate leads to the reduced production of PSA.

The treatment by androgen suppression alone for early stage prostate cancer can induce the biochemical cure by decreasing the PSA level to a nadir value of 0. 1 ng per ml. Routinely, such PSA measurements is used to assess the success of treatment by external beam or interstitial radiation for early stage prostate cancer (2; Carroll P. R. et.al, Cancer of the Prostate, In Cancer, Principles and Practice of Oncology, 6^{th} edition, Vol. 1; DeVita, Jr. et al (Ed), 2001 page 1455 (Ref. # 535)) Patients with advanced prostate cancer, androgen suppression treatment can also lower the serum PSA to a nadir value of 0.1 ng per ml.

The treatment of prostate cancer and its relative prognosis is better defined and discussed in terms of its stage at the time of diagnosis. In the following descriptions on the diagnosis and treatment of prostate cancer, the American Joint Committee on Cancer, AJCC cancer staging system of before 1998 is elected. The present commonly used staging system is the 1998 modified AJC cancer staging system. However since the literature that are to be referred here in the discussions relates to 10, 15 and to 20 year post treatment survival, the older AJC staging system is the more relevant one for the discussions here. The newer methods of interstitial radioactive seed implant treatment of T0-T2 prostate cancer have no 10 to 15-year survival data. For the available 3 and 5 year results for interstitial radioactive seed implants and that are referred here, no efforts to correct the small differences in stages T0-T2 between before 1998 and since 1988 AJC staging system is made.

The American Joint Committee on Cancer, AJCC cancer staging system of before 1998 defines the primary prostate cancer based upon the extent of the disease and as the following:

The T1 tumor is incidental histologic finding. The T1 tumor is further sub-classified as:

T-1a: three or more microscopic foci

T1b: More than three microscopic foci

T2: tumor is present clinically or grossly and limited to the prostate.

T2a: tumor less than 1.5 cm, with normal tissue in at least three sides.

T2b: tumor greater than 1.5 cm or in more than one lobe

In T3 category, the tumor invades the prostatic apex or into or beyond the prostatic capsule, balder neck or seminal vesicle, but is not fixed

T4: pelvic fixation

There is no consensus on the best form of treatment for early stage prostate cancer. The present major methods of treatment of early stage prostate cancer includes surgical prostatectomy, external beam radiation therapy, prostate implants by radioactive sources, namely brachytherapy, and cryotherapy namely freezing the prostate with cryosurgical devices. The hormonal treatments aimed at androgen depravation, chemotherapy and palliative treatments with bisphosphonates and radiopharmaceuticals are generally reserved for patients with advanced diseases.

Before the PSA era detection and treatment of early stage, T0-T2 prostate cancer by immediate or delayed androgen depravation therapy gave 81 per cent corrected survival at 15 years (3; Carroll P. R. et.al, Cancer of the Prostate, In Cancer, Principles and Practice of Oncology, 6^{th} edition, Vol. 1; DeVita, Jr. et al (Ed), 2001 page 1439) It is very close to the fifteen-year survival reported for surgery and external radiation therapy. There is no such comparable fifteen-year survival for interstitial radiation therapy by the improved trans rectal ultrasound guided implant methods. The older methods of interstitial radioactive seed implants gave much inferior tumor control as compared to surgery or external beam radiation therapy (4,5,6; Carroll P. R. et.al, Cancer of the Prostate, In Cancer, Principles and Practice of Oncology, 6^{th} edition, Vol. 1; DeVita, Jr. et al (Ed), 2001 page 1454 (Ref. 529, 530, 532)

Presently, the early stage prostate cancer is routinely treated either by surgery, external radiation therapy or by interstitial radioactive seed implants. The corrected 81 per cent fifteen year survival of patients with stage T0-T2 and treated by immediate or delayed androgen suppressive treatment necessitates its comparison with fifteen year survival for patients with similar stage T0-T2 and treated by surgery or radiation therapy. There are no such comparable fifteen-year survivals for interstitial radiation therapy by present improved method consisting of transrectal ultrasound aided radioactive seed implants and hence it cannot be included in such a comparison.

The 10-15 year overall and disease specific survival of patients with stage T1- and T2 prostate cancer and treated by immediate or delayed androgen suppressive treatment ranged 62 to 90 per cent, which is very close to those of age-matched men from the general population. The risk of developing metastasis in such conservatively treated patients at 10 and 15 years were 13 to 20 per cent for patients with T1-T2 disease (3,7, 8; Carroll P. R. et.al, Cancer of the Prostate, In Cancer, Principles and Practice of Oncology, 6^{th} edition, Vol. 1; DeVita, Jr. et al (Ed), 2001 pages 1439 (Ref. # 288), page 1493, (Ref. # 285 and 287))

The 10-year crude and cause specific survival rates of patients with localized disease (T0-T2) after radical prostatectomy is 75 and 90 per cent (9; Carroll P. R. et.al, Cancer of the Prostate, In Cancer, Principles and Practice of Oncology, 6^{th} edition, Vol. 1; DeVita, Jr. et al (Ed), 2001 pages 1442, (Ref. # 307) and 10; Rosh III, M., Wallner, K., Prostate Cancer, In Textbook of Radiation Oncology; Liebel, S A, Phillips, T L, (Ed), 1998, p768-769, table 42-11). The crude survival rate of about 75 per cent at 10 and 15 years after radical prostatectomy for patients with clinically localized disease is like those of age-matched men from the general population (9; Carroll P. R. et.al, Cancer of the Prostate, In Cancer, Principles and Practice of Oncology, 6^{th} edition, Vol. 1; DeVita, Jr. et al (Ed), 2001 pages 1442, (Ref. # 307)) Using an undetectable PSA as an endpoint for failure, only about 50 per cent of patients are disease free at 10 years after radical prostatectomy (10; Rosh III, M., Wallner, K., Prostate Cancer, In Textbook of Radiation Oncology; Liebel, S A, Phillips, T L, (Ed), 1998, p768-769, table 42-11).

The 10-year cause specific mortality of patients with stage T1-T2 prostate cancer and treated by radiation therapy in the RTOG trail 77-06 and reported Hanks et al was 14 per cent. In other words, the cause specific survival at 10 years for this group of patients was 86% (11,12; Carroll P. R. et.al, Cancer of the Prostate, In Cancer, Principles and Practice of Oncology, 6^{th} edition, Vol. 1; DeVita, Jr. et al (Ed), 2001 page 1452, (Ref. # 411), page 1493, (Ref. # 505)). This survival rate is almost like those of age-matched men from the general population. The observed age matched survival rate at 10 years for this group of patients with T1b-T2 prostate cancer was 63 per cent. Before the 1998 modification of the staging system defined the T1b prostate cancer as the one with more than three microscopic foci, the T2a as a tumor less than 1.5 cm, with normal tissue on at least three sides and the T2b tumor greater than 1.5 cm or in more than one lobe. As per lymphadenectomy, all the 104 patients in this group with T1b-T2 tumor had no lymph node metastasis. It is very close to the expected age matched 59 per cent survival at 10 years (11,12; Carroll P. R. et.al, Cancer of the Prostate, In Cancer, Principles and Practice of Oncology, 6^{th} edition, Vol. 1; DeVita, Jr. et al (Ed), 2001 page 1452, (Ref. # 411), and page 1493, (Ref. # 505)).

The long-term survival of patients with localized prostate cancer treated conservatively by either observation or immediate or delayed androgen suppression correlates well with the tumor differentiation. Presently, the Gleason tumor grading system, a sum of two most microscopic appearances is widely used to define the tumor grading. The loss of life at 15 years for patients with Gleason score 2 to 4, well differentiated tumor and conservatively treated by observation alone or by immediate or late androgen suppression is well within the expected life expectancy for general population. Compared to general population, the loss of life at 15 years for patients with Gleason score of 5 to 7 and treated conservatively by observation alone or by immediate or delayed androgen suppression are 4 to 5 years. Similar comparison with the general population on the expected loss of life at 15 years for patients with Gleason score of 8 to 10 prostate cancer by observation and or androgen suppression treatment is 8 to 10 years. (13; Carroll P. R. et.al, Cancer of the Prostate, In Cancer, Principles and Practice of Oncology, 6^{th} edition, Vol. 1; DeVita, Jr. et al (Ed), 2001 page 1440, (Ref. # 290)). If patients with Gleason score 2 to 4 were treated conservatively by immediate or delayed androgen suppression the probability of dying from prostate cancer at 15 years is 7 per cent. If the Gleason scores were 5, the probability of dying with prostate cancer at fifteen years is 6 to 11 per cent. Patients with Gleason scores 6 and if they are treated by immediate or delayed androgen suppression treatment only, their chances of dying with prostate cancer would increase to 18 to 30 per cent. If patients with Gleason score 8, the poorly differentiated prostate cancer, elects to have immediate or delayed androgen suppression treatment only, their chances of dying from prostate cancer at 15 years would be 42 to 70 per cent. If patients with Gleason score of 10, the most poorly differentiated and prognostically the worst group of patients, their chances of dying at 15 years would increase to 60 to 87 per cent. (14; Carroll P. R. et.al, Cancer of the Prostate, In Cancer, Principles and Practice of Oncology, 6^{th} edition, Vol. 1; DeVita, Jr. et al (Ed), 2001 page 1440, (Ref. # 299)).

In summary, T0-T2 prostate cancer treated by immediate or delayed androgen depravation therapy is reported as 81 per cent corrected 15-year survival (3) and 62 to 90 per cent ten to fifteen year overall and disease specific survival (3,15,16) It is very close to the 10 and 15-year survival of patients with comparably staged prostate cancer and treated by surgery or radiation therapy. The 10-year crude and cause specific survival rates for patients with localized disease (T0-T2) after radical prostatectomy is 75 and 90 per cent (9,10). The 10-year cause specific mortality of patients with stage T1-T2 prostate cancer and treated by radiation therapy in the RTOG trail 77-06 and reported Hanks et al was 14 per cent. In other words, the cause specific survival at 10 years for this group of patients was 86% (11,12; Carroll P. R. et.al, Cancer of the Prostate, In Cancer, Principles and Practice of Oncology, 6^{th} edition, Vol. 1; DeVita, Jr. et al (Ed), 2001 page 1452, (Ref. # 411) and page 1493, (Ref. # 505)).

The overall and cause specific survival of 62 to 90 per cent at 10 to 15 years (3,15,16) and the corrected survival 81 per cent at 15 years by conservative primary hormonal treatment result for stage T0-T2 prostate cancer could be further improved by improved patients selection criteria for such treatment and delivery of androgen suppressive steroidal hormones to the prostate at sufficiently high concentrations but with lesser or no systemic toxicity (3 Carroll P. R. et.al, Cancer of the Prostate, In Cancer, Principles and Practice of Oncology, 6^{th} edition, Vol. 1; DeVita, Jr. et al (Ed), 2001 page 1439, (Ref. # 288)). Like the T stage, the degree of tumor differentiation also determines the ultimate clinical course treatment outcome for prostate cancer. If patients were selected for primary hormonal treatment on the basis of better prognostic Gleason grade of 2 to 6 and stage T0-T2 at diagnosis, the above overall and cause specific survival of 62 to 90 per cent at 10 to 15 years (3,15,16; Carroll P. R. et.al, Cancer of the Prostate, In Cancer, Principles and Practice of Oncology, 6^{th} edition, Vol. 1; DeVita, Jr. et al (Ed), 2001 page 1439, (Ref. # 288) and page 1440, (Ref # 289 and 298)) and corrected survival of 81 per cent at 15 years (3; Carroll P. R. et.al, Cancer of the Prostate, In Cancer, Principles and Practice of Oncology, 6^{th} edition, Vol. 1; DeVita, Jr. et al (Ed), 2001 page 1439, (Ref. # 288)) would further improve. Such a selection criterion is followed for interstitial radioactive seed implant treatment for early stage prostate cancer. The other important feature is that it will preserve the potency. Potency following radical prostatectomy or radiation therapy is significantly reduced. It would be a more convenient treatment to a patient with early stage prostate cancer. Furthermore, it would reduce the cost of treatment for early stage prostate cancer significantly.

The two thirds of the prostate cancer occurs in men aged seventy and over and it has a history of long slow growth and clinical course of many years. An improved conservative hormonal treatment for early stage T0-T2 well to moderately differentiated, Gleason scores 2 to 6 prostate cancer that would facilitate near tumor control as with surgery and radiation therapy lends the logical opportunity for deferred treatment by surgery or radiation therapy. A treatment policy of primary hormonal treatment and watchful waiting until clinical and or biochemical evidence of disease progression is noticed for elective surgery or radiation therapy is appropriate for this chronic disease of the elderly men.

For early stage prostate cancer, many forms of treatments are available; however there is no consensus on the best form of such treatment. Because of the easily available PSA testing many more very early stage prostate cancers are detected. Such early detected early stage prostate cancers falls into either the low or intermediate risks group or to the high-risk group. Immediate and aggressive treatment may not be necessary in some of these patients (17; Carroll P. R. et.al, Cancer of the Prostate, In Cancer, Principles and Practice of Oncology, 6^{th} edition, Vol. 1; DeVita, Jr. et al (Ed), 2001 page 1435). Many of the low risk group patients may be managed by high efficiency hormone implants alone. Patients with high-risk early stage prostate cancer may also benefit from hormone implant alone or the hormone implant combined with surgery, external radiation therapy combined with interstitial radioactive seed implants or by interstitial radioactive seed implants.

The 10 to 15 year overall and disease specific survival for patients treated conservatively by androgen suppression is 62 to 90 per cent and that for stage T3 and T4 disease, it is 57 to 70 per cent (3,15,16; Carroll P. R. et.al, Cancer of the Prostate, In Cancer, Principles and Practice of Oncology, 6^{th} edition, Vol. 1; DeVita, Jr. et al (Ed), 2001 page 1439, (Ref. # 288) and page 1440, (Ref. # 289 and 298)). The risk of local and metastatic progression and ultimate death also correlates with the T staging of the prostate cancer. For patients with early stage T1-T2 prostate cancer at diagnosis, the chances of developing metastatic progression at 10 to 15 years after diagnosis is 13 to 20 per cent. Ten per cent of patents with T1a tumor and 47 per cent of patients with T1b tumor would ultimately die of prostate cancer. The risk of local and metastatic progression for patients with T2 tumor is like that of patients with stage T1 tumor, namely 13 to 20 per cent at 10 to 15 years. The chances of dying from prostate cancer for patients with stage T2a and T2b disease at diagnosis are 52 and 53 per cents respectively. For patients with T3 tumor at diagnosis and treated by conservative hormonal management, their chances of metastatic tumor progression at 10 to 15 years is about 25 to 34 years and ultimately dying due to prostate cancer is 53 per cent. Seventy per cent of patients with T4 tumor at diagnosis will die of prostate cancer (18; Carroll P. R. et.al, Cancer of the Prostate, In Cancer, Principles and Practice of Oncology, 6^{th} edition, Vol. 1; DeVita, Jr. et al (Ed), 2001 page 1439, (Ref. # 291)).

Because of the abundance of androgen receptors in an undisturbed early stage prostate cancer, it would be more amenable to androgen suppression treatment than after radiation treatment of an early stage prostate cancer. The cytotoxic effects of radiation to the prostate would remove or diminish the available androgen receptor sites and hence the androgen suppression treatment of early stage prostate cancer after radiation would not be as effective as when treatment is given before radiation. Both radiation and surgery are equally effective in the treatment of early stage prostate cancer. After treatment of prostate cancer by radical prostatectomy, there is no prostate and therefore no postoperative androgen suppression treatment is needed. Hence, the locally implanted androgen suppressive hormonal compositions would be more effective before radiation treatment of early stage prostate cancer.

Treatments by surgery, external beam radiation, interstitial seed implants with radioactive seeds, all are effective to induce either complete or partial cure of prostate cancer. It is evidenced by absent serum PSA after radical prostatectomy or the PSA level reaching to a nadir value of about 0.1 ng per ml after radiation therapy. After successful radical prostatectomy, serum PSA is undetectable. Presence of postoperative PSA is considered as still present residual prostate tissue and as a biochemical failure. After external beam or interstitial implant radiation treatment of patients with early stage prostate cancer, a sustained PSA level of not more than 1 ng per ml for five years is suggestive of 95 per cent likelihood of permanent tumor control (19; Carroll P. R. et.al, Cancer of the Prostate, In Cancer, Principles and Practice of Oncology, 6^{th} edition, Vol. 1; DeVita, Jr. et al (Ed), 2001 page 1447). An elevated PSA after such treatment is indicative of residual tumor and or tumor recurrence. The same low PSA level will be observed after androgen suppressive treatment as by treatment with external beam radiation and or interstitial radioactive seed implants. Even patients with serum PSA levels exceeding over 300 ng per ml, treatment with 1 mg DES three times a day would reduce the PSA to less than 1 ng per ml. However presently, the androgen suppressive treatment alone is not an elective routine treatment for early stage prostate cancer.

The routine PSA testing of males allows the very early biochemical detection of prostate cancer including those in their formative very early stage. Even those with apparent normal serum PSA level but with an increase in PSA dynamic ratio could be an early indication of evolving early stage prostate cancer. Pre PSA period T0-T2 prostate cancer treated by immediate or delayed androgen deprivation therapy had 81 per cent corrected survival at fifteen years (3; Carroll P. R. et.al, Cancer of the Prostate, In Cancer, Principles and Practice of Oncology, 6^{th} edition, Vol. 1; DeVita, Jr. et al (Ed), 2001 page 1439, (Ref. # 288)). It is almost identical to patents similarly staged and treated by surgery, radioactive seed implants or external beam radiation. The radiation therapy by radioactive seed implants has no such comparable 10 or 15 year survival. A 93 per cent 3-year PSA based biochemical tumor control has been reported for implant treatment with I-125 pr Pd-103 (20; Rosh III, M., Wallner, K., Prostate Cancer, In Textbook of Radiation Oncology; Liebel, S A, Phillips, T L, (Ed), 1998, p768, (Ref. # 127)). In the biology of prostate cancer, it is a short period of follow up. Because of the patients selection criteria of early stage low grade and low PSA for radioactive seed implant, the reported 3 year 93 per cent tumor control for such patients is thought to be an optimistic estimate (21; Rosh III, M., Wallner, K., Prostate Cancer, In Textbook of Radiation Oncology; Liebel, S A, Phillips, T L, (Ed), 1998, p768). Early stage T1-T2, good and favorable prognostic group with Gleason score 2 to 6 based patient selection is used for the elective radioactive seed implant treatment of prostate cancer (22; Rosh III, M., Wallner, K., Prostate Cancer, In Textbook of Radiation Oncology; Liebel, S A, Phillips, T L, (Ed), 1998, p763-764). The method of patient selection for radioactive seed implant treatment can significantly effect the treatment results. (22; Rosh III, M., Wallner, K., Prostate Cancer, In Textbook of Radiation Oncology; Liebel, S A, Phillips, T L, (Ed), 1998, p763-764).

Radioactive seed implant brachytherapy is discouraged for patients with poorly differentiated tumor that is high grade Gleason score, PSA greater than 20 ng per ml and extensive bilateral disease in biopsy specimen (22; Rosh III, M., Wallner, K., Prostate Cancer, In Textbook of Radiation Oncology; Liebel, S A, Phillips, T L, (Ed), 1998, p763-764).

The PSA based very early-detected prostate cancer staged as T0-T2 and with well to moderately differentiated tumor, PSA 20 ng per ml or less is selected for radioactive seed implant treatment. If patients are similarly selected for primary androgen suppressive hormonal implant treatment as for the radioactive seed implant the biochemical and long-term tumor control would not differ much for each form of these treatments. The reported 81 per cent corrected survival at fifteen years by the immediate or delayed androgen deprivation therapy (3; Carroll P. R. et.al, Cancer of the Prostate, In Cancer, Principles and Practice of Oncology, 6^{th} edition, Vol. 1; DeVita, Jr. et al (Ed), 2001 page 1439, (Ref. # 288)) would be further increased by such more efficient but lesser toxic androgen deprivation therapy alone. The T0-T2a well-differentiated (Gleason 4) to moderately differentiated (Gleason 5 and 6) tumors and undisturbed by aggressive treatments such as radiation will have more androgen receptor sites in it. Therefore, the locally implanted androgen depriving hormonal compositions will inhibit the tumor cell division more efficiently. It will induce early proliferative tumor cell death and the biochemical tumor control evidenced by the normal serum PSA level.

The tumor control probability by radiation therapy alone is shown as a linear function of the radiation dose. The tumor positive biopsies decreases to about 52, 25 and 5.4 per cent, at the radiation doses of 64.8 Gy, 75.6 Gy, and 81 Gy reduced the positive biopsies to 6 per cent (23,24; Carroll P. R. et.al, Cancer of the Prostate, In Cancer, Principles and Practice of Oncology, 6^{th} edition, Vol. 1; DeVita, Jr. et al (Ed), 2001 page 1453, (Ref. # 415) and page 1454, (Ref. # 520)) which is equivalent to the negative biopsies observed by 81 Gy external radiation therapy alone.

The ability of external radiation therapy combined with androgen suppressive treatment to reduce the volume of the prostate and the rate of post treatment positive biopsies is now well established (25; Rosh III, M., Wallner, K., Prostate Cancer, In Textbook of Radiation Oncology; Liebel, S A, Phillips, T L, (Ed), 1998, p777, Table 42-23). This beneficial effect of combined radiation and pre and post radiation androgen suppression was shown in a randomized trial by Laverdiere et al in 1997 (26, Rosh III, M., Wallner, K., Prostate Cancer, In Textbook of Radiation Oncology; Liebel, S A, Phillips, T L, (Ed), 1998, p777, Table 42-23, (Ref. # 297). In this randomized study, Laverdiere et al demonstrated that when stage T2 and T3 prostate cancer was treated by conventional external radiation therapy to 64 Gy, there were 67 per cent tumor positive biopsies at 12 months and 69 per cent at 24 months. If the patients were treated by total androgen suppression (TAS) with LHRH and flutamide for three months before radiation, the positive biopsies were 36 per cent at 12 months and 29 per cent at 24 months. When the treatment with TAS was started three months before radiation and continued for six more months after the radiation, the positive biopsies at 12 months was 8.7 per cent and at 24 months just 6 per cent. It is an excellent example for the effectiveness the androgen suppressive treatment to control both the early stage T2 and higher stage T3 prostate cancer and its ability to reduce the toxicity associated with high dose radiation therapy by combining the lower dose radiation with androgen deprivation hormonal therapy. Significantly improved local tumor control, disease free survival, time to development of distant metastasis and an improvement in overall survival at 5 years for poor prognostic group patients with high grade locally advanced disease was also reported for combined radiation and androgen suppressive treatment

(27; Rosh III, M., Wallner, K., Prostate Cancer, In Textbook of Radiation Oncology; Liebel, S A, Phillips, T L, (Ed), 1998, p778 (Ref. # 28)). When radiation therapy was combined with androgen suppressive treatment, better tumor control including improved local tumor control, freedom from metastasis and PSA failure was also reported by the Radiation Oncology Group studies (28, 29; Rosh III, M., Wallner, K., Prostate Cancer, In Textbook of Radiation Oncology; Liebel, S A, Phillips, T L, (Ed), 1998, p776-777, Table 42-23, (Ref. # 301 and 280)).

Concomitant hormonal and radiation treatment is much lesser toxic and would be well tolerated. Androgen suppressive hormonal implants to prostate before and after the radiation therapy either as conventional external or interstitial radiation would nearly sterilize all the focus of tumor. High efficiency androgen suppressive hormonal treatment with its higher prostatic and lower systemic concentration that is just sufficient to suppress the hypothalamic LHRH and pituitary LH and FSH secretion but with minimal systemic toxicity would further improve the tumor control that was reported by Laverdiere et al. It would improve both the biochemical and the clinical tumor control. The slow-release long-term hormonal implants would minimize both the clinical and the biochemical failures. It would maintain lower levels of serum PSA and acid phosphatase for several years than by the short duration systemic hormonal administration as was used in the study of Laverdiere et al.

The primary combined radiation and androgen suppressive hormonal treatment would be more appropriate for patients with biologically aggressive tumor as evident form the Gleason grading, T stage and serum PSA and acid phosphatase levels at diagnosis.

The conservative management of early stage T0-T2 prostate cancer by androgen suppression treatment alone will not eliminate all the focus of tumor. Interference with androgen signaling pathways will generate proliferative arrest and or to differentiate the cancer cells into a phenotype that is less malignant and to programmed, apoptotic cell death. However, after androgen suppression alone treatment for early stage prostate cancer, the biopsy specimen might still contain tumor cells. If after androgen suppressive hormone implant treatment, tumor growth becomes evident as by an increase of PSA, digital examination or by imaging studies, then additional treatment with radiation or surgery may be selected. For those patients who may have no tumor progression after androgen suppressive implant treatment, no additional treatment may be needed.

It is known that androgen suppression reduces the size of the prostate gland. Androgen suppressive treatment is routinely used to reduce the size of a bulky prostate gland and to make it more amenable for interstitial implants with radioactive seeds. The androgen suppression treatment alone would also reduce the tumor associated elevation of PSA to a normal level. Patients with bulky T2-b-T4 tumors when treated by androgen suppression combined with radiation there were better local and biochemical tumor control. It was evidenced by lower PSA failures as compared to the group similarly treated but without androgen suppression treatment (29; Rosh III, M., Wallner, K., Prostate Cancer, In Textbook of Radiation Oncology; Liebel, S A, Phillips, T L, (Ed), 1998, p776-777, Table 42-23, Ref. # 280)). A valid criticism against such biochemical tumor control by combined radiation and androgen suppressive treatment is that the androgen suppression alone would bring the serum PSA level to normal.

Among the natural and synthetic estrogens used to treat patients with prostate cancer include diethylstilbestrol (DES), chlorotrianisene (Tace), diethylstilbestrol diphosphate, polyestradiol phosphate, and ethinyl estradiol (30, Jensen E V, Estrogen binding and clinical response of breast cancer: In Cancer Medicine; Holland J. F., Frei III, E. (Ed), 1974, page 922.). These estrogenic hormones were widely used for the treatment of advanced prostate cancer. High dose estrogen treatment is associated with systemic toxicity like thromboembolism and disturbance in the lipid metabolism. Hence presently, the advanced prostate cancer is treated with safer, but definitively more expensive hormonal compositions like the LHRH agonists.

In U.S. Pat. No. 4,321,208 (31; Sahadevan V: Preparation of directly iodinated steroid hormones and related compounds, U.S. Pat. No. 4,321,208; 1982) this inventor has described the methods for preparation of iodinated steroid hormones including the estradiol as early as in 1976, the time of filing the patent application. The I-125 labeled estradiol was also shown to bind to estrogen antiserum and to the estrogen receptor sites. Likewise, the competitive binding of I-125 labeled estradiol to its receptor sites by DES was also shown by this inventor in U.S. Pat. No. 4,321,208 (31, Sahadevan V: Preparation of directly iodinated steroid hormones and related compounds, U.S. Pat. No. 4,321,208; 1982) Because of the heaviness and the electronegative characteristic of iodine in the estradiol molecule, it would render also its cytotoxic actions to the prostate cancer during this deiodination process. Implantation iodoestradiol adsorbed sponges to rat breast tumor showed excellent tumor regression (unpublished data).

The present methods of androgen deprivation therapy of prostate cancer consist of administration of a LHRH analogue alone or with an anti-androgen compound. The long-acting depot preparations of LHRH analogue is injected subcutaneousely and the anti-androgen and or steroids are administrated as oral preparations. In addition to the higher cost of LHRH analogues, like the anti-androgens, it has numerous side effects.

The side effects of anti-androgens and LHRH include hot flash, fatigue, gynecomastia, decrease in muscle and red cell mass, reduction and or loss of libido, thromboembolism and cardiac deaths. Because of these side effects, especially due to thromboembolism and the occasional cardiac deaths associated with estrogens like the DES, presently the oral and the injectable form of estrogens are not used as part of hormonal treatment of prostate cancer. Like the very high cost LHRH analogues, the very low-cost estrogens can also block the hypothalamic LHRH secretion, which in turn blocks the pituitary LH and FSH secretion resulting in the diminished and or no synthesis of testicular androgens.

With the hope to minimize the systemic toxicity of estrogenic hormones like the DES, compositions like diethylstilbestrol diphosphate and polyestradiol phosphate were developed. It was thought that this phosphate containing estrogenic hormones would accumulate more in the prostate due to hydrolysis of the phosphate group by prostatic acid phosphatase. The prostate contains acid phosphatase and it is significantly increased in prostate cancer. However, only a fraction of the systemically administered diethylstilbestrol diphosphate will reach the prostate and deposited as diethylstilbestrol. A relatively increased prostatic uptake of intravenously or orally administered high doses of diethylstilbestrol diphosphate in the prostate and associated better tumor control was reported (30; Jensen E V, Estrogen binding and clinical response of breast cancer: In Cancer Medicine; Holland J. F., Frei III, E. (Ed), 1974, page 922). However, high doses of intravenous or oral diethylstilbestrol diphosphate have also systemic toxicity. Because of the tonicities associated with the treatments of prostate cancer with such estrogen and its synthetic derivatives, they are no more used to treat the prostate cancer.

The present hormonal treatment of prostate cancer is primarily with the peptide analogues of leuteinizing hormone releasing hormone (LHRH) that has both partial agonistic and antagonistic affects. The commercially available LHRH preparations, the leuprolide and goserelin are partial agonists that initially stimulate the hypothalamic secretion of the leuteinizing hormone (LH) and the follicle stimulating hormone (FSH) followed by inhibition of the hypothalamic secretion of the LH and FSH. Because of the inhibition of the LH and FSH secretion, the LH-FSH-dependent testicular testosterone synthesis is also inhibited. Like the commercial preparations of LHRH analogues, the leuprolide and the goserelin, estrogens also inhibit the hypothalamic secretions of LH and FSH. These LHRH analogues have equal effectiveness as estrogen or orchiectomy to inhibit the hypothalamic LH, FSH secretion. The preparations of the LHRH analogues, the leuprolide and the goserelin are very expensive. The cost for a three months duration depot preparation of leuprolide is about $1,500. For a years supply of this hormone will cost about $6,000. Estrogens are much less costly and they are readily available. The cost for a year's daily estrogen supply for a patient's treatment for prostate cancer is about $500 or less. It is about 1,200 per cent less than the cost of a year's supply of a LHRH analogue. However, because of the side effects of systemically administered estrogens, it is not commonly used.

A controlled slow release implant of a depot preparation of estrogen directly to the prostate could achieve high concentrations of estrogens to the prostate and its very low concentration in the rest of the body. This low systemic estrogen would be sufficient to inhibit the hypothalamic LHRH mediated pituitary LH-FSH secretion. Estrogen toxicity is reduced and or eliminated by the very low levels of systemic estrogen. The high levels of estrogen from the estrogen implants to the prostate would saturate the estrogen receptors of the prostate. It would enhance the effectiveness of the estrogen treatment of prostate cancer. Furthermore, such hormone implants can also be combined with anti-androgen compounds that bind to androgen receptor sites of the prostate and blocks the androgen binding to the prostate. It would further enhance the effectiveness of the hormonal treatment of prostate cancer. The slow-release combination hormone implants therapy for prostate cancer is a much less invasive treatment.

Because of the systemic distribution of the orally administered or injected estrogens and anti-androgen compounds, only a portion of these compounds will reach the intended target site, the prostate. In addition, the methods of oral and or injectable forms of anti-androgen administration need more disciplined compliance by the patients to take these medications daily or periodically. Furthermore, a greater percentage of such systemically distributed compounds are metabolized. Therefore, much larger doses of these compounds are needed to insure the delivery of the required dose at the target site, the prostate. The commonly available pharmaceutical preparation of Depo-Provera containing medroxyprogesterone is used for contraceptive treatment. A subcutaneous implant of an oily preparation of 150 mg of medroxyprogesterone will provide 1 to 7 ng of medroxyprogesterone per ml plasma for three months (32; Pharmacia and Upjohn Company, Depo-Provera, Physicians Desk Reference, PDR, 51,1997,p2079). Implantation of steroid pellets under the skin is a well-known method of treatment with hormones.

Injections of pellets of hormones for hormone replacement treatment after oophorectomy result in large variations in serum hormone levels with high levels immediately after such injections. Hence the generally known methods of preparation of injectable slow-release depot formulations of hormones encapsulated in biodegradable polymers is made to deliver a constant dose of hormone. Similar preparations of microcapsules were described in U.S. Pat. No. 4,389,330 (33; Tice T R, and Lewis D H: Microencapsulation process, U.S. Pat. No. 4,389,330; 1983). Similar preparations are referenced and described in U.S. Pat. No. 5,340,586 (34; Pike M and Spicer D V: Methods and formulations for use in treating oophorectomized women, U.S. Pat. No. 5,340,586; 1994). Injectable encapsulated hormone preparations are made to facilitate a steady state of hormone release for periods ranging from a few days to several years and are used as subcutaneous injections for the hormone replacement treatment after oophorectomy U.S. Pat. No. 5,340,586 (34; Pike M and Spicer D V: Methods and formulations for use in treating oophorectomized women, U.S. Pat. No. 5,340,586; 1994).

Several methods of preparation of pellets of compounds of steroids and other compositions are known in the art, which dates back as early as 1936 and onwards. Several of these methods are cited in the U.S. Pat. No. 4,244,949 (35; Gupta G N: Manufacture of long term contraceptive implant, U.S. Pat. No. 4,244,949; 1981) of 22 years ago, the entire disclosure of which is hereby incorporated by reference. In a preferred art for such implants preparation, the steroid is mixed with a lipoid carrier consisting of cholesterol and its organic carboxylic esters and loading and compacting this mixture into a Teflon tubing and heating the tubing at a temperature above the melting point of the steroid and lipoid under an inert gas like nitrogen, cooling the tubing and removing the pellets of fused steroid-lipoid composition. Cholesterol serves as the lipoid carrier. This formulation facilitates the constant slow release of desired dose of steroid hormone from the implanted bioabsorbable fused steroid-lipoid composition. Examples of such constant release implants of steroid hormones to provide 50 to 80 μg steroid per day in rhesus monkey is given in U.S. Pat. No. 4,244,949 (35; Gupta G N: Manufacture of long term contraceptive implant, U.S. Pat. No. 4,244,949; 1981) and which is sufficient to achieve the contraceptive effects of such formulation for one year and more in rhesus monkeys.

The U.S. Pat. No. 4,244,949 (35; Gupta G N: Manufacture of long term contraceptive implant, U.S. Pat. No. 4,244,949; 1981) uses the bioabsorbable fusion products of anti-ovulation steroid hormone and a lipoid carrier selected from the group of cholesterol for making the slow-release long acting contraceptives. Preparations of fusion products of steroid and lipoid were well known in the prior art, 23 years ago when this patent application was made. As claimed in this patent, the fused implant was made for fertility control and not as either by subcutaneous or intramuscular injections or by direct implant to the prostate for the hormonal treatment of prostate cancer.

The methods of preparations of encapsulated hormone implants described in U.S. Pat. No. 5,430,585 (36; Pike M and Spicer D V: Methods and formulations for use in treating benign gynecological disorders; U.S. Pat. Nos. 5,340,585; 1994) and 5,430,586 (34; Pike M and Spicer D V: Methods and formulations for use in treating oophorectomized women, U.S. Pat. No. 5,340,586; 1994) were also known in the prior art. Those prior art methods are discussed and referenced in these patents. U.S. Pat. No. 5,430,585 (36; Pike M and Spicer D V: Methods and formulations for use in treating benign gynecological disorders; U.S. Pat. No. 5,340,585; 1994) teaches methods and formulations of treatment of benign gynecological disorders and the U.S. Pat. No. 5,340,586 (34; Pike M and Spicer D V: Methods and formulations for use in treating oophorectomized women, U.S. Pat. No. 5,340,586; 1994) teaches the methods and formulations for treatment of oophorectomized women. They do not teach the treatment of prostate cancer either by subcutaneous or intramuscular injections or by direct prostate implants of those encapsulated and or microspheres preparations of hormones. Furthermore, the hormonal compositions of the implant preparations of U.S. Pat. No. 5,430,585 (36; Pike M and Spicer D V: Methods and formulations for use in treating benign gynecological disorders; U.S. Pat. Nos. 5,340,585; 1994) and 5,430,856 (34; Pike M and Spicer D V: Methods and formulations for use in treating oophorectomized women, U.S. Pat. No. 5,340,586; 1994) containing androgen are not suitable for the androgen suppressive treatment of prostate cancer. The steroid hormonal compositions of androgen and estrogen encapsulated in Silastic silicone tube implants were used for male contraception in U.S. Pat. No. 4,210,644 (37; Ewing L L, Desjardins C: Male contraception; U.S. Pat. No. 4,210,644; 1980). Androgen is a growth stimulant of prostate cancer. Suppression of androgen production is the primary goal of hormonal treatment of prostate cancer. Hence its composition is not suitable for the treatment of prostate cancer. In the present invention described in this application, similar encapsulation methods are used to make implantable suitable hormonal compositions for the treatment of prostate cancer.

Like in U.S. Pat. No. 4,210,644 (37; Ewing L L, Desjardins C: Male contraception; U.S. Pat. No. 4,210,644; 1980), the long acting synthetic progestin, the levonorgestrel encapsulated in Silastic silicone rubber tubing is used to prepare the Norplant System of Wyeth—Ayerst Laboratory's long-acting contraceptive (43; Norplant System, Wyeth Ayerst Laboratories, Physicians Desk Reference, PDR, 51, 1997, p2868). Implantation of this long acting encapsulated contraceptive levonorgestrel protects from fertility up to 5 years. These implants are usually implanted subcutaneousely to the upper arm. After 5 years, the inert and empty Silastic capsule is removed from the implant site. This is also not intended for the treatment of prostate cancer. Progestin is not very effective in the treatment of prostate cancer as the estrogenic compounds like DES.

The U.S. Pat. No. 6,326,467 (38; Nett T M, Glode L M, Wieczorek M and Jarosz P J: Hormone-recombinant toxin compounds and methods for using same; U.S. Pat. No. 6,326,467; 2001), the entire disclosure of which is hereby incorporated by reference, teaches the preparation of LHRH-recombinant toxin compounds and its use to sterilize animals and for the treatment of estrogen dependent breast cancer and androgen dependent prostate cancer. This is achieved by means of destruction of the LHRH dependent pituitary LH and FSH secreting cells by these compounds and thereby inhibiting the LH and FSH dependent estrogen production in the ovary and androgen production in the testis. It is administrated by intravenous, subcutaneous or intramuscular injections. This patent also gives a review of other methods used to inhibit the LHRH action on LH and FSH secreting pituitary cells. It includes descriptions of prior art protein toxin conjugated LHRH as a vaccine to produce antibodies directed against LHRH and thus to neutralize the endogenous LHRH and thereby deprive the stimulation of the pituitary LH and FSH secreting cells to produce LH and FSH. In the absence of LH and FSH the production of estrogens and androgens by the gonads are inhibited.

The above U.S. Pat. No. 6,326,467 (38; Nett T M, Glode L M, Wieczorek M and Jarosz P J: Hormone-recombinant toxin compounds and methods for using same; U.S. Pat. No. 6,326,467; 2001) teaches the use of LHRH-recombinant toxin to replace the use of DES in the treatment of prostate cancer. DES is known to be effective in hormone-refractory prostate cancer (39; Carroll P. R., Lee, K. L., Fuks, Z. Y., Kantoff, P. W., Cancer of the Prostate, In Cancer, Principles and Practice of Oncology, 6^{th} edition, Vol.1; DeVita, Jr., Hellman. S, and Rosenberg, (Ed), 2001, p.1464, (Ref. 651)). Therefore, the beneficial actions of DES in the treatment of prostate cancer is not entirely similar to the inhibition of LHRH, LH and FSH pathways associated androgen production to deprive the androgens to androgen dependent prostate cancer. While LHRH analogous acts as an agonist and antagonist and is effective in the treatment of androgen dependent prostate tumors, the effectiveness of DES treatment of prostate cancer including in hormone-refractory prostate cancer indicates that DES has other cytotoxic actions than just being an estrogen. Thus the direct implants of DES to a tumor bearing prostate gland has many more beneficial tumor controlling actions than the LHRH treatment of prostate cancer. The local direct slow release DES implants alone or implants containing DES in combination with an anti-androgen compound that competes with androgen for the androgen binding receptor sites of the prostate cancer and or with other cytotoxic agents would be more effective in the treatment of prostate cancer than the treatment of prostate cancer with LHRH analogues or by ablation of pituitary FSH and LH secreting cells with LHRH-recombinant toxin. Furthermore, such direct implants of DES, anti-androgens and cytotoxic compounds are much more cost effective.

U.S. Pat. No. 6,248,057 (40; Mavity W G, Stern R A, Osaki S, and Zamora P O: Absorbable brachytherapy and chemotherapy delivery devices and methods; U.S. Pat. No. 6,248,057; 2001), the entire disclosure of which is hereby incorporated by reference, is aimed at a modified form of interstitial radioactive seed implant, the brachytherapy but with cytotoxic agents incorporated into the implant. The Greek word “brachy” meaning “short range” is coined to describe the “short range” radiation therapy, namely the brachytherapy by implanting short-range radioactive sources either into a tissue or to a body cavity. When the short-range radioactive seeds are implanted directly into a tumor, it is termed as interstitial brachytherapy. When the radioactive sources are placed in a body cavity that is in close proximity to a tumor, it is called intracavitary brachytherapy. The effectiveness of radioactive sources to induce tissue damage was recognized immediately after the discovery of radioactivity by Antoine Henri Becquerel, isolation of radium by Marie Curie in 1898 and by the very first radiobiological experiments of Pierre Curie in 1901. Soon after the turn of the century, Alexander Graham Bell suggested implanting radioactive sources directly into a tumor. It is one of the methods of treatment for highly selected early stage prostate cancer. However such treatment has many proponents and critics.

The U.S. Pat. No. 6,248,057 (40; Mavity W G, Stern R A, Osaki S, and Zamora P O: Absorbable brachytherapy and chemotherapy delivery devices and methods; U.S. Pat. No. 6,248,057; 2001) suggests a modification of this century old practice of brachytherapy by attaching a chemotherapeutic agent to the radioactive seeds to facilitate combined local radiation and chemotherapy. For radiation protection and to facilitate dose calculation per geometry, the radioactive seeds are always encapsulated in strong metallic capsules. In U.S. Pat. No. 6,248,057 (40; Mavity W G, Stern R A, Osaki S, and Zamora P O: Absorbable brachytherapy and chemotherapy delivery devices and methods; U.S. Pat. No. 6,248,057; 2001) such encapsulations are thought to be a disadvantage. It is said that these implants would cause difficulties in future diagnostic interventions; however such difficulties are very seldom in clinical practice. With the intent not to distribute the radioactivity systemically and to facilitate the local radiation and chemotherapy at the same time, this patent describes process and methods for the radioactive element and the drug to be adsorbed on to the walls of the bioabsorbable implant structures. They are made to stay as adsorbed or bound to the implant structure's wall until they are released by biodegradation. Here the intent is to keep the radioactivity localized as in classical brachytherapy while facilitating local delivery of chemotherapeutic drugs during the course of short duration radiation emission from the radioactive sources by biodegradation of the implants.

By replacing the classical methods of encapsulation of the radioactive isotopes in metallic tubing of titanium, platinum or gold with bioabsorbable structures are used for the preparation of radioactive seeds for brachytherapy, the advantages of the absorption of unwanted energies of α and β emission is lost. It also brings significant dosemetric difficulties. The clinical dose of brachytherapy is currently referenced to a useful formalism. It depends on the assumption that the radioactive source is an axially symmetric source of active length L with dose rate in a plane through the source axis at an arbitrary distance r from the source center and an angle 2° with respect to axis (41; Anderson L L, Weaver K A, Physics of Brachytherapy, In Textbook of Radiation Oncology; Liebel, S A, Phillips, T L, (Ed).,1998, p 156 ). Such dosimetric formalism cannot be applied to the bioabsorbable structures with radioactivity diffusely distributed as coated or attached to the implant structures. This will cause significant dosimetric difficulties. The dose rates and energy of many of the radioactive isotopes that are suggested to coat the biodegradable implant structures in U.S. Pat. No. 6,248,057 (40; Mavity W G, Stern R A, Osaki S, and Zamora P O: Absorbable brachytherapy and chemotherapy delivery devices and methods; U.S. Pat. No. 6,248,057; 2001) are another major dosimetric and radiobiological difficulties.

Preparation of bioabsorbable drug compositions adsorbed to a carrier and the methods of labeling of bioabsorbable compositions is well known in the prior arts. Some of those prior arts are cited in this patent. In one model of commonly used iodine-125 seed implants, the iodine is adsorbed on to ion exchange resin and it is then encapsulated into a titanium tube (42; Anderson L L, Weaver K A, Physics of Brachytherapy, In Textbook of Radiation Oncology; Liebel, S A, Phillips, T L, (Ed).,1998, p 152). If there were no titanium tubing encapsulation, this iodine-125 adsorbed ion exchange resin also would be bioabsorbed.

This patent claim that the drug composition of this bioabsorbable implants is released with in two half-lives of the radionuclide. Repeatedly it is being stated in this patent that the “chemotherapeutic drug is released during the persistence period of radioactivity”. It is intended to achieve the therapeutic effectiveness of concomitant radiation and chemotherapy. Short-lived isotopes are used to prepare such bioabsorbable implants. Long-lived isotopes like iodine-125 have 60 days half-life. When such long-lived isotopes are used for local radiation and it is combined with long periods of localized chemotherapy, it can cause numerous complications including local carcinogenesis itself. If iodine-125 bioabsorbable implants were made to absorb earlier, its systemic toxicity would be prohibitive because of the high radioactivity released systemically. In prostate implant treatment with iodine-125, the cumulative dose to the prostate is about 160 Gy (16000 rads). If a significant portion of this dose were released into the circulation from biodegradation of radioactive implants, it would be a very toxic dose. Hence, the suitable isotopes cited as examples for the preparation of bioabsorbable implants in U.S. Pat. No. 6,248,057 (40; Mavity W G, Stern R A, Osaki S, and Zamora P O: Absorbable brachytherapy and chemotherapy delivery devices and methods; U.S. Pat. No. 6,248,057; 2001) have half-lives ranging from 9.8 min. to 3.8 days. Two half-lives of such suitable isotopes therefore will have a range of 19.6 min. to 7.6 days. Therefore the drug composition released from these bioabsorbable implants last for only about 7.6 days at the most. It is not suitable for hormonal treatment of cancer, particularly for the prostate cancer. As described earlier, the androgen suppressive treatment maintains a constant level of serum androgen suppressive hormone for several months to years. to effect the suppression of the androgen synthesis. The persistence periods of the bioabsorbable structures of U.S. Pat. No. 6,248,057 is claimed as 2 to 90 days (40; Mavity W G, Stern R A, Osaki S, and Zamora P O: Absorbable brachytherapy and chemotherapy delivery devices and methods; U.S. Pat. No. 6,248,057; 2001). The maximum persistence period of 90 day is only 1.5 times half-life of iodine-125. Therefore, if iodine-125 and a hormone containing bioabsorbable implants according to this U.S. Pat. No. 6,248,057 (40; Mavity W G, Stern R A, Osaki S, and Zamora P O: Absorbable brachytherapy and chemotherapy delivery devices and methods; U.S. Pat. No. 6,248,057; 2001) were elected for prostatic implants in spite of all its other disadvantages, it is still a very short period of androgen suppressive treatment. Furthermore if the usual I-125 implant radiation dose of 160 Gy were attempted with bioabsorbable implants of U.S. Pat. No. 6,248,057 (40; Mavity W G, Stern R A, Osaki S, and Zamora P O: Absorbable brachytherapy and chemotherapy delivery devices and methods; U.S. Pat. No. 6,248,057; 2001), then in 90 days of its maximum persistence, the radioactivity released from its biodegradation would severely harm the patient. The I-125's half-life of 60 days would makes it impossible to treat the prostate cancer with the interstitial brachytherapy and chemotherapy implants of maximum 90 days persistence as in U.S. Pat. No. 6,248,057 (40; Mavity W G, Stern R A, Osaki S, and Zamora P O: Absorbable brachytherapy and chemotherapy delivery devices and methods; U.S. Pat. No. 6,248,057; 2001). It is the same case if other long-lived isotopes were substituted in lieu of I-125 to prepare combined interstitial brachytherapy and chemotherapy. In spite of this potential clinical difficulties associated with long lived isotopes having up to 60 days half-live, the U.S. Pat. No. 6,248,057 (40; Mavity W G, Stern R A, Osaki S, and Zamora P O: Absorbable brachytherapy and chemotherapy delivery devices and methods; U.S. Pat. No. 6,248,057; 2001) claims the radionuclides incorporated in to its implantable biodegradable structures having half life up to 60 days.

All the drug formulations included in the U.S. Pat. No. 6,248,057 (40; Mavity W G, Stern R A, Osaki S, and Zamora P O: Absorbable brachytherapy and chemotherapy delivery devices and methods; U.S. Pat. No. 6,248,057; 2001) are for combined interstitial radiation and chemotherapy. There are no teachings on the primary or secondary hormonal treatment of the prostate cancer in U.S. Pat. No. 6,248,057 (40; Mavity W G, Stern R A, Osaki S, and Zamora P O: Absorbable brachytherapy and chemotherapy delivery devices and methods; U.S. Pat. No. 6,248,057; 2001). In this U.S. Pat. No. 6,248,057 (40; Mavity W G, Stern R A, Osaki S, and Zamora P O: Absorbable brachytherapy and chemotherapy delivery devices and methods; U.S. Pat. No. 6,248,057; 2001), there are no teachings on steroidal hormonal treatment for cancer, including for prostate cancer. The known anti-neoplastic chemotherapeutic drugs are incorporated into these biodegradable implants. As a general statement, it is said that the natural or synthetic peptide hormones could be included in the group of drug compositions that can be combined with the short duration interstitial brachytherapy described in U.S. Pat. No. 6,248,057 but without any specifications or examples. Here again it is only a statement that is supportive of the intended very short duration drug delivery system described in U.S. Pat. No. 6,248,057 (40; Mavity W G, Stern R A, Osaki S, and Zamora P O: Absorbable brachytherapy and chemotherapy delivery devices and methods; U.S. Pat. No. 6,248,057; 2001) intended to enhance the effectiveness of the interstitial radiation therapy. Furthermore, in the case of peptide hormones like the LHRH agonists, its therapeutic effectiveness on prostate cancer is mediated through the hypothalamic pituitary axis. Subcutaneous, intramuscular or intravenous injections are the usual routes of administration of peptide hormones. Their injection directly to a site like the prostate has no clinical advantages than by the above routes of administration.

Drugs like daunorubicin and doxorubicin are also included in the drug formulary of these implants. Locally released daunorubicin and doxorubicin by biodegradation of such implants would cause severe local reaction such as tissue necrosis and associated serious consequences. In clinical practice, severe tissue necrosis from a very small amount of this drug's infiltration to their intravenous site of administration is a much-feared local reaction. Together with radiation, such local tissue reaction can be even more severe. Hormonal implants to prostrate for the treatment of early stage prostate cancer is a much more benign form of treatment. It has much lesser toxicity.

### OBJECTS AND ADVANTAGES

It is therefore, an object of this invention to provide a less or no toxic improved method of primary hormonal treatment of early stage, low and intermediate risk prostate cancers than the treatment of said disease by surgery or radiation therapy and the more complex and expensive surgery or radiation therapy is reserved for patients failing to respond to said primary hormonal treatment comprising of prostatic implants of steroid hormones in one or more slow release formulations and permitting said drugs to be continuously released at near constant rate directly to the prostate for longer periods and maintaining said formulation's serum level sufficient to effect suppression of androgen synthesis but low enough to minimize or to eliminate systemic toxicity.

It is another object of the invention to provide slow-release biodegradable seeds or microcapsules or Silastic capsules containing androgen suppressive hormonal formulations for prostatic implant methods for the primary hormonal treatment of prognostically favorable early stage prostate cancer and said treatment as an alternative to the more complex methods of treatment by surgery or radiation therapy.

Another object of the invention is to provide slow-release prostatic hormonal implant products for treating prostate cancer with less toxicity and cost as an alternative to other presently available treatment for prognostically favorable early stage prostate cancer and said methods consisting of implanting biodegradable seeds or microcapsules or Silastic capsules containing said hormone formulations to deliver high concentrations of said hormonal formulations to the prostate for longer periods.

Still another object of this invention is to provide high concentrations of androgen suppressive formulations in the prostate by said formulation's direct implant in the gland which obviates the necessity of daily systemic administration in higher doses for the treatment of prostate cancer.

It is a further object of this invention to maintain high concentrations of androgen suppressive formulations in the prostate by implanting slow-release biodegradable seeds or microcapsules or Silastic capsules containing androgen suppressive hormonal formulations for prostatic implant to maintain such formulation's systemic concentration low by dilution of said released formulations through circulation and thereby eliminate or minimize the systemic toxicity associated with such formulations.

It is still a further object of this invention to maintain high concentrations of androgen suppressive formulations in the prostate by implanting slow-release biodegradable seeds or microcapsules or Silastic capsules containing androgen suppressive hormonal formulations and to follow up the biochemical tumor response to said treatment by periodic estimations of serum prostate specific antigen, and acid phosphatase.

It is still another object of this invention to make implants of cytotoxic drugs alone or in combination with androgen suppressive compositions as slow-release longer lasting biodegradable seeds or microcapsules or Silastic capsules containing said compositions for prostatic implants to deliver high concentrations of said formulations to the prostate for extended periods and to inhibit the hypothalamic LHRH, pituitary FSH and LH secretions by its systemic circulation thereby inhibit testosterone production for a predetermined longer period as an efficient treatment of prostate cancer with lesser toxicity than by said formulation's higher dose systemic administration by oral, subcutaneous or intramuscular routes.

It is still another object of this invention to make implants of androgen suppressive compositions as slow-release longer lasting biodegradable microcapsules containing said compositions for prostatic implants in injectable mediums which make chelating composition when said formulation comes in contact with prostatic tissue and thereby delivering high concentrations of said formulations to the prostate for an extended period and inhibit hypothalamic LHRH, pituitary FSH and LH secretions by its systemic circulation and thereby inhibit the testosterone production for a predetermined extended period as an efficient treatment of prostate cancer with lesser toxicity than by said formulation's higher dose systemic administration by oral, subcutaneous or intramuscular routes.

It is still another object of this invention to make implants of natural estrogens and their synthetic derivatives alone or in combination with other androgen suppressive compositions as slow-release biodegradable seeds or microcapsules or Silastic capsules containing said compositions for prostatic implants to deliver high concentrations of said formulations to the prostate and to inhibit the hypothalamic LHRH, pituitary FSH and LH secretions by its systemic circulation and thereby inhibit testosterone production as an efficient hormonal treatment of prostate cancer with lesser toxicity than by said formulation's higher dose systemic administration by oral, subcutaneous or intramuscular routes.

It is a further object of this invention to make implants of iodo-estradiol as slow-release biodegradable seeds or microcapsules or Silastic capsules containing said compositions for prostatic implants to deliver high concentrations of said formulations to the prostate and to inhibit the hypothalamic LHRH, pituitary FSH and LH secretions by its systemic circulation and thereby inhibit testosterone production as an efficient hormonal treatment of prostate cancer with lesser toxicity than by said formulation's higher dose systemic administration by oral, subcutaneous or intramuscular routes.

It is another object of this invention to make implants of anti-androgen flutamide, bicalutamide or nilutamide alone or in combination with other androgen suppressive compositions as slow-release biodegradable seeds or microcapsules or Silastic capsules containing said compositions for prostatic implants to deliver high concentrations of said formulations to the prostate and to inhibit the hypothalamic LHRH, pituitary FSH and LH secretions by its systemic circulation and thereby inhibit testosterone production as an efficient hormonal treatment of prostate cancer with lesser toxicity than by said formulation's higher dose systemic administration by oral, subcutaneous or intramuscular routes.

It is still another object of this invention to make implants of natural corticosteroids and their synthetic derivatives alone or in combination with other androgen suppressive compositions as slow-release biodegradable seeds or microcapsules or Silastic capsules containing said compositions for prostatic implants to deliver high concentrations of said formulations to the prostate for the treatment of hormone refractory prostate cancer and to inhibit the hypothalamic LHRH, pituitary FSH and LH secretions by its systemic circulation and thereby inhibit testosterone production as an efficient hormonal treatment of prostate cancer with lesser toxicity than by said formulation's higher dose systemic administration by oral, subcutaneous or intramuscular routes.

Still it is another object of this invention to make implants of estramustine alone or in combination with other androgen suppressive compositions as slow-release biodegradable seeds or microcapsules or Silastic capsules containing said compositions for prostatic implants to deliver high concentrations of said formulations to the prostate for the treatment of hormone dependent and hormone refractory prostate cancer and to inhibit the hypothalamic LHRH, pituitary FSH and LH secretions by its systemic circulation and thereby inhibit testosterone production as an efficient hormonal treatment of prostate cancer with lesser toxicity than by said formulation's higher dose systemic administration by oral, subcutaneous or intramuscular routes.

It is a further object of this invention to make implants of DES and its derivatives alone or in combination with other androgen suppressive compositions as slow-release biodegradable seeds or microcapsules or Silastic capsules containing said compositions for prostatic implants to deliver high concentrations of said formulations to the prostate for the treatment of hormone dependent and hormone refractory prostate cancer and to inhibit the hypothalamic LHRH, pituitary FSH and LH secretions by its systemic circulation and thereby inhibit testosterone production as an efficient hormonal treatment of prostate cancer with lesser toxicity than by said formulation's higher dose systemic administration by oral, subcutaneous or intramuscular routes.

It is still a further object of this invention to make implants of natural progesterone and its synthetic derivatives alone or in combination with other androgen suppressive compositions as slow-release biodegradable seeds or microcapsules or Silastic capsules containing said compositions for prostatic implants to deliver high concentrations of said formulations to the prostate and to inhibit the hypothalamic LHRH, pituitary FSH and LH secretions by its systemic circulation and thereby inhibit testosterone production as an efficient hormonal treatment of prostate cancer with lesser toxicity than by said formulation's higher dose systemic administration by oral, subcutaneous or intramuscular routes.

It is a further object of this invention to make prostate implants of steroidal and non-steroidal anti-androgen compounds as fused with a lipoid carrier, or as injectable microcapsules or encapsulated in Silastic capsules to achieve slow release of said compounds by diffusion and biodegradation of the carrier or by diffusion alone and the slowly released anti-androgen to bind and to saturate the prostatic androgen receptor sites competitively with androgens to block the growth and proliferation of the prostate cancer with lesser systemic toxicity than by said compound's daily high dose systemic administration.

It is another object of this invention to make prostate implants of natural and synthetic estrogens fused with a lipoid carrier, or as injectable microcapsules or encapsulated in Silastic capsules to achieve slow release of said compounds by diffusion and biodegradation of the carrier or by diffusion alone and for the slowly released estrogens to bind and to saturate the prostatic estrogen receptor sites competitively with androgens to block the growth and proliferation of the prostate cancer with lesser systemic toxicity than by said compound's daily high dose systemic administration.

It is still another object of this invention to make implants of androgen suppressive compositions as slow-release biodegradable seeds or microcapsules or Silastic capsules containing said compositions for implants to gross metastatic prostate cancer to deliver high concentrations of said formulations to the metastasis and to inhibit the hypothalamic LHRH, pituitary FSH and LH secretions by its systemic circulation and thereby inhibit testosterone production as an efficient hormonal treatment of hormone dependent and refractory metastasis of the prostate cancer with lesser toxicity than by said formulation's higher dose systemic administration by oral, subcutaneous or intramuscular routes.

It is a further object of this invention to reduce the cost of present hormonal treatment of prostate cancer substantially by direct prostatic implants of long acting steroidal and non-steroidal hormones, anti-androgen compounds and to increase the efficiency of such treatments but with lesser toxicity than by such compounds daily systemic administration.

A further object of this invention is to minimize or to eliminate side effects such as thromboembolic events associated with treatments of prostate cancer with estrogens by minimizing its systemic concentration and maximizing its prostatic contents by implanting said implants directly to the prostate and allowing slow release of such compositions from the implants to the prostate by diffusion and biodegradation.

A further object of this invention is to minimize or to eliminate the side effects of anti-androgen treatments of prostate cancer such as fatigue, gynecomastia, decrease in muscle and red cell masses, bone loss, and decreased body hair due to the toxic effects of the contents of such prostatic implants by maintaining its low systemic dose and its high prostatic contents by release of the contents of said implants directly to the prostate by diffusion and biodegradation.

It is another object of this invention to make implants of cytotoxic drugs alone or in combination with androgen suppressive compositions as slow-release longer lasting biodegradable seeds or microcapsules or Silastic capsules containing said compositions for prostatic implants to deliver high concentrations of said formulations to the prostate for an extended period as part of concomitant radiation and hormonal treatment and to inhibit the hypothalamic LHRH, pituitary FSH and LH secretions by its systemic circulation and thereby inhibit testosterone production as an efficient hormonal and cytotoxic treatment of prostate cancer with lesser toxicity than by said formulation's higher dose systemic administration by oral, subcutaneous or intramuscular routes.

It is still another object of this invention to make implants of androgen suppressive compositions as slow-release longer lasting biodegradable seeds or microcapsules or Silastic capsules containing said compositions for prostatic implants and maintaining of said drug compositions for extended periods by diffusion and biodegradation from said prostatic implants at an amount effective to suppress focal tumor development as prophylaxis and to follow up of any evidence of potential tumor development by periodic estimations of serum prostate specific antigen and to suppress testicular and adrenal androgen synthesis as a prophylaxis towards the development of prostate cancer with minimum or no systemic toxicity.

Other objects, together with the foregoing are attained in the exercise of the method described in the following description and resulting in the embodiment illustrated in the accompanying drawing.

Still further objects and advantages will become apparent from a consideration of the ensuing description and accompanying drawings

### BRIEF DESCRIPTION OF THE DRAWINGS

Other objects and advantages of this invention will become more apparent from the specification taken in conjunction with the accompanying drawings, in which:

FIG. 1 shows the immediate inducement of biochemical tumor control of the prostate cancer by treatment with DES as evidenced by its effectiveness to decrease serum prostate specific antigen (PSA) from its pretreatment very high value of 363 ng per ml to a normal level rapidly.

FIG. 2 illustrate the long-term effective biochemical tumor control of the prostate cancer by treatment with DES as evidenced by the decrease in the pretreatment very high PSA value of 363 ng per ml to less than 0.1 ng and maintaining it at this low nadir value for four years.

FIG. 3 shows the immediate inducement of biochemical tumor control by treatment with DES as evidenced by its effectiveness to decrease serum acid phosphatase from its pretreatment very high value of 48 international units per ml to a normal level rapidly.

### DETAILED DESCRIPTION OF THE DRAWINGS

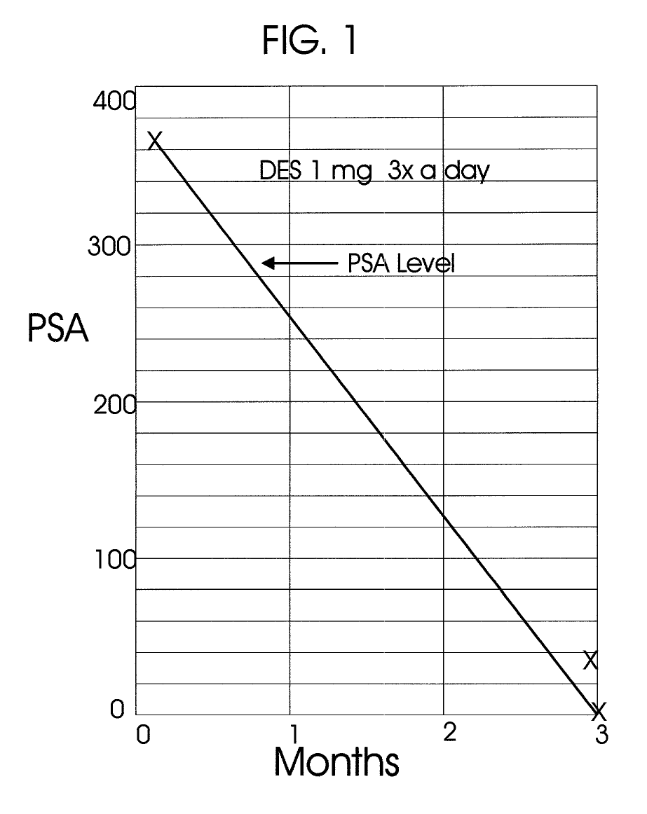

In FIG. 1, the rapid decrease of serum PSA of a patient with recurrent poor prognostic prostate cancer with grossly elevated PSA and acid phosphatase by secondary hormonal treatment with DES is illustrated. Since this hormonal treatment was rendered at recurrence after radiation therapy, it is termed as secondary hormonal treatment. A rapidly increasing serum PSA level indicated the biochemical failure of tumor control by the initial radiation therapy. Before the treatment was started with DES 1 mg three times a day, the serum PSA has reached to 363 ng per ml. Three months after treating with DES 1 mg three times a day, the serum PSA level has decreased first to 38.4 ng per ml and then a near normal value of 4.5 ng per ml.

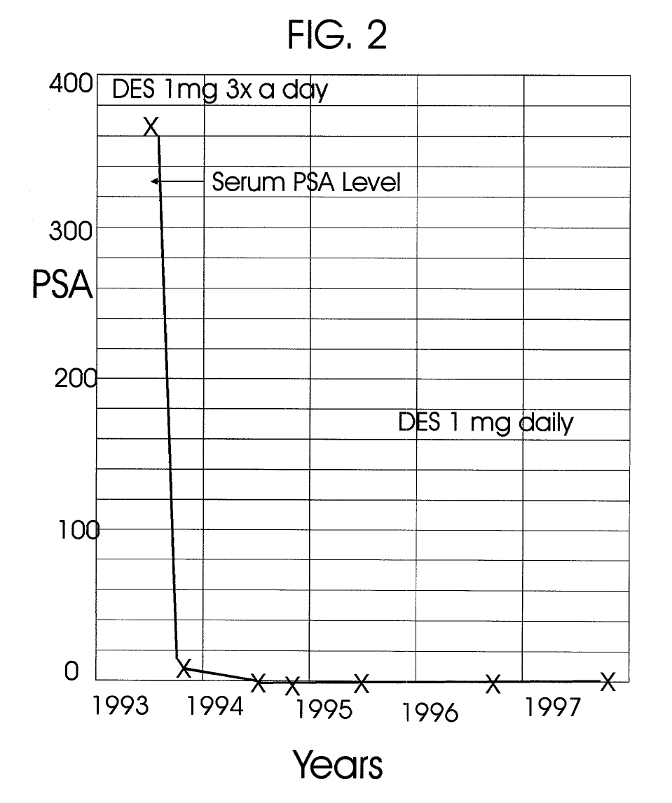

The FIG. 2 illustrates the effectiveness of continued treatment with DES to maintain a low nadir serum PSA levels in the same patient described in FIG. 1. The secondary hormonal treatment with DES alone was continued and without any other added cytotoxic agents, radiation therapy or surgery. The initial dose of DES was 1 mg three times a day. Later it was reduced to 1 mg per day. This secondary hormonal treatment with DES has induced a total biochemical tumor control as evidenced by the decrease in the pretreatment very high PSA value of 363 ng per ml to less than 0.1 ng and maintaining it at this low nadir value for four years.

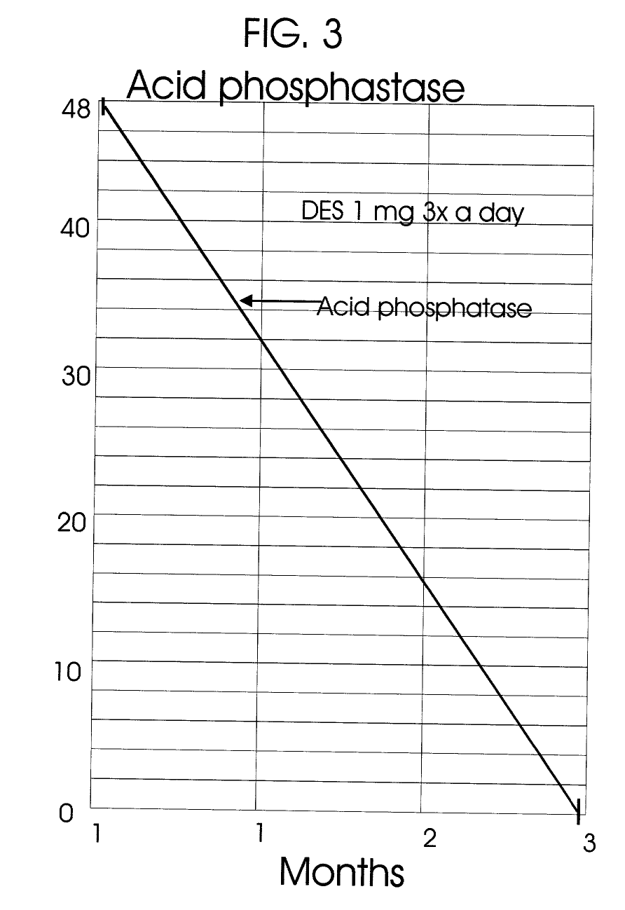

In FIG. 3, the rapid decrease of serum acid phosphatase of the same patient with recurrent prostate cancer and grossly elevated PSA and acid phosphatase by the secondary hormonal treatment with DES is illustrated. A rapidly increasing serum acid phosphatase level indicated the biochemical failure of tumor control by the initial radiation therapy. Before the treatment was started with DES 1 mg three times a day, the serum acid phosphatase has reached to a very high value of 48.0 international units per ml. Like the PSA, the serum acid phosphatase has also decreased very rapidly. Three months after treating with DES 1 mg three times a day, the serum acid phosphatase level has decreased to less than the normal value of 0.3 international units per ml.

FIGS. 1-3 illustrates the effectiveness of the orally administered DES to induce biochemical tumor control even in poor prognostic group of patients with recurrent prostate cancer. The data contained in FIGS. 1-3 are summarized in the Table below:

| | Daily DES | PSA | Acid |
|---|---|---|---|
| Date | dose | ng/ml | Phosphatase iu |
| June 10, 1993 | 3 mg 3× daily | 363.0 | 48.0 |
| September 7, 1993 | 3 mg 3× daily | 38.4 | |
| September 15, 1993 | 3 mg 3× daily | 4.5 | 0.3 |
| June 13, 1994 | 3 mg 3× daily | >0.1 | |
| September 26, 1994 | 3 mg 3× daily | >0.1 | |
| May 3, 1995 | 3 mg 3× daily | >0.1 | |
| August 4, 1996 | 1 mg daily | >0.1 | |
| August 5, 1997 | 1 mg daily | >0.1 | |

After external beam or interstitial implant radiation treatment of patients with early stage prostate cancer, a sustained PSA level of not more than 1 ng per ml for five years is suggestive of 95 per cent likelihood of permanent tumor control (19; Carroll P. R. et.al, Cancer of the Prostate, In Cancer, Principles and Practice of Oncology, 6^{th} edition, Vol. 1; DeVita, Jr. et al (Ed), 2001 page 1447). An elevated PSA after such treatment is indicative of residual tumor and or tumor recurrence. The same low PSA level is observed by per oral administration of androgen suppressive treatment as by treatment with external beam radiation and or interstitial radioactive seed implant. In this instance, this patient had serum PSA levels of 363 ng per ml before the treatment with DES was started. Even DES at an oral dose of 1 mg daily is shown to reduce the PSA to 0.1 ng per ml. However, the biochemical tumor control alone is not an indication of absolute tumor control for poor prognostic recurrent prostate cancer.

The 93 per cent 3-year PSA based biochemical tumor control is reported for I-125 or Pd-103 base interstitial implant treatment (20; Rosh III, M., Wallner, K., Prostate Cancer, In Textbook of Radiation Oncology; Liebel, S A, Phillips, T L, (Ed), 1998, p768, (ref. # 127). In the biology of prostate cancer, it is a short period of follow up. Because of the patient selection criteria of early stage low grade and low PSA for radioactive seed implant, the reported 3 year 93 per cent tumor control for such patients is thought to be an optimistic estimate (21; Rosh III, M., Wallner, K., Prostate Cancer, In Textbook of Radiation Oncology; Liebel, S A, Phillips, T L, (Ed), 1998, p768). Early stage T1-T2, good and favorable prognostic group patients with Gleason scores 2 to 6 are selected for the elective radioactive seed implant treatment of prostate cancer (22; Rosh III, M., Wallner, K., Prostate Cancer, In Textbook of Radiation Oncology; Liebel, S A, Phillips, T L, (Ed), 1998, p763-764). Radioactive seed implant brachytherapy is discouraged for patients with poorly differentiated tumor, that is those with Gleason score higher than 6, PSA greater than 20 ng per ml or those with extensive disease present in both lobes of the prostate per biopsy (22; Rosh III, M., Wallner, K., Prostate Cancer, In Textbook of Radiation Oncology; Liebel, S A, Phillips, T L, (Ed), 1998, p763-764). This patient had PSA up to 363 ng per ml and extensive tumor in both lobes of the prostate. Because of the frequent tumor extension to the pelvic and abdominal lymph nodes, such unfavorable disease could not be controlled even by external beam radiation therapy. Such cancer of the prostate will not also be controlled by interstitial radiation therapy. In this instance, DES 1 mg three times per day by mouth showed excellent early induction of biochemical tumor control. It was effective to maintain the biochemical tumor control for four years of follow-ups. During this period of treatment with DES, the serum PSA and the acid phosphatase remained at a nadir value of 0.1 ng per ml and 0.3 international unit respectively. However, the biochemical tumor control alone is not an indication of absolute tumor control for poor prognostic recurrent prostate cancer.

The locally implanted androgen depriving hormonal compositions would be even more effective to induce early proliferative tumor cell death evidenced by the normal serum PSA as the biochemical tumor control than by the systemically administered androgen suppressive agents like the oral DES administration. Such implants will be as effective as surgery or radiation therapy for the treatment of good prognostic early stage prostate cancer. Furthermore, it will maintain more sexual potency. It is much less toxic and it is a simpler, less expensive treatment than by surgery or radiation therapy. The more invasive complex and expensive surgery or radiation therapy is reserved for patients failing the primary hormonal androgen suppressive formulation's prostatic implant treatment.

### SUMMARY

Two thirds of prostate cancers occurs in men age seventy and over and it has a history of long slow growth and a clinical course of many years. The overall cause specific survival of patients with stage T0-T2 prostate cancer and treated conservatively by immediate or delayed androgen suppressive treatment is 62 to 90 per cent at 10 to 15 years and their corrected 15 year survival is 81 per cent. It is very close to those of similarly staged patients treated by surgery or radiation therapy and that of age-matched men from the general population. An improved lesser toxic, conservative androgen suppressive treatment for patients with stage T0-T2, well to moderately differentiated, Gleason scores 2 to 6 prostate cancers. Because of the abundance of androgen receptors in an undisturbed early stage prostate cancer, it would be more amenable to androgen suppression treatment than after its treatment with radiation. Patients are similarly selected for the interstitial radioactive seed implant, brachy therapy.

A treatment policy of primary hormonal treatment and watchful waiting until clinical and/or biochemical evidence of disease progression is taken into consideration before elective surgery or radiation therapy for this chronic disease of the elderly men. The important feature is that it will preserve potency more than by the alternative treatments of surgery or radiation therapy. Potency following radical prostatectomy or radiation therapy is significantly reduced. It would be a more convenient treatment to a patient with early stage prostate cancer. Furthermore, it would reduce the cost of treatment for early stage prostate cancer significantly.

Because of the systemic distribution of the orally administered or injected estrogens and anti-androgen compounds, only a portion of these compounds will reach the intended target site, the prostate. A greater percentage of such systemically distributed compounds are metabolized. Therefore, much larger doses of these compounds are taken daily or very frequently to insure the delivery of the required dose to the prostate, which increases its systemic toxicity and the cost.

Prostatic implants of androgen suppressive drugs formulated as fused with a lipoid carrier or encapsulated in microcapsules or in Silastic capsules render a constant slow-release of their contents to the prostate for extended periods by biodegradation and diffusion. They facilitate higher prostatic and lower systemic concentrations of androgen suppressive hormones. Because of their high concentrations in the prostate, tumor control is much more improved. Their lower systemic concentrations are maintained sufficient to suppress hypothalamic LHRH, pituitary LH and FSH mediated testicular and adrenal androgen synthesis. Because of these lower systemic concentrations, their toxicity is minimized or eliminated. These slow-release of androgen suppressive compositions from the extended period implants minimize both clinical and the biochemical failures. It would maintain lower levels of serum PSA and acid phosphatase for several years

Localized prostate cancer is well controlled by concomitant hormonal and conventional dose radiation treatment as by treatment with higher dose radiation without the hormonal treatment. Lower dose conventional radiation combined with androgen suppressive treatment is a much less toxic treatment than the higher dose radiation alone.

Androgen suppressive hormonal implants to the prostate before, during or after radiation therapy would nearly sterilize all the focus of the tumor. They would maintain lower levels of serum PSA and acid phosphatase for several years.

Two thirds of prostate cancers occurs in men age seventy and over and it has a history of long slow growth and a clinical course of many years. The overall cause specific survival of patients with stage T0-T2 prostate cancer and treated conservatively by immediate or delayed androgen suppressive treatment is 62 to 90 per cent at 10 to 15 years and their corrected 15 year survival is 81 per cent. It is very close to those of similarly staged patients treated by surgery or radiation therapy and that of age-matched men from the general population. An improved lesser toxic, conservative androgen suppressive treatment for patients with stage T0-T2, well to moderately differentiated, Gleason scores 2 to 6 prostate cancers. Because of the abundance of androgen receptors in an undisturbed early stage prostate cancer, it would be more amenable to androgen suppression treatment than after its treatment with radiation. Patients are similarly selected for the interstitial radioactive seed implant, brachy therapy.

A treatment policy of primary hormonal treatment and watchful waiting until clinical and/or biochemical evidence of disease progression is taken into consideration before elective surgery or radiation therapy for this chronic disease of the elderly men. The important feature is that it will preserve potency more than by the alternative treatments of surgery or radiation therapy. Potency following radical prostatectomy or radiation therapy is significantly reduced. It would be a more convenient treatment to a patient with early stage prostate cancer. Furthermore, it would reduce the cost of treatment for early stage prostate cancer significantly.

Because of the systemic distribution of the orally administered or injected estrogens and anti-androgen compounds, only a portion of these compounds will reach the intended target site, the prostate. A greater percentage of such systemically distributed compounds are metabolized. Therefore, much larger doses of these compounds are taken daily or very frequently to insure the delivery of the required dose to the prostate, which increases its systemic toxicity and the cost.

Prostatic implants of androgen suppressive drugs formulated as fused with a lipoid carrier or encapsulated in microcapsules or in Silastic capsules render a constant slow-release of their contents to the prostate for extended periods by biodegradation and diffusion. They facilitate higher prostatic and lower systemic concentrations of androgen suppressive hormones. Because of their high concentrations in the prostate, tumor control is much more improved Their lower systemic concentrations are maintained sufficient to suppress hypothalamic LHRH, pituitary LH and FSH mediated testicular and adrenal androgen synthesis. Because of these lower systemic concentrations, their toxicity is minimized or eliminated. These slow-release of androgen suppressive compositions from the extended period implants minimize both clinical and the biochemical failures. It would maintain lower levels of serum PSA and acid phosphatase for several years Localized prostate cancer is well controlled by concomitant hormonal and conventional dose radiation treatment as by treatment with higher dose radiation without the hormonal treatment. Lower dose conventional radiation combined with androgen suppressive treatment is a much less toxic treatment than the higher dose radiation alone. Androgen suppressive hormonal implants to the prostate before during or after radiation therapy would nearly sterilize all the focus of the tumor. They would maintain lower levels of serum PSA and acid phosphatase for several years.

### DETAILED DESCRIPTION OF THE INVENTION

Pursuant to the present invention, the method of prostate cancer treatment by androgen suppressive and cytotoxic compositions is improved by direct prostatic implants of such composition's depot formulations. The therapeutic effectiveness of such depot formulation is significantly improved by maintaining such formulation's higher concentration in the prostate. Because of its systemic dilution, its serum concentration is much low. The serum level of such compositions is kept as to suppress the hypothalamic LHRH mediated pituitary LH and FSH secretions and thereby to inhibit androgen synthesis. This low-level systemic concentration of the androgen suppressive compounds such as the estrogens and or anti-androgens diminishes and or eliminates many of the side effects associated with their daily oral, or frequent intravenous, intramuscular or subcutaneous administration. The direct prostatic implants of androgen suppressive compositions facilitate complete saturation of its binding sites in the prostate. It is a much more efficient treatment than by their administration by oral, intravenous, intramuscular or subcutaneous routes.

A number of methods for preparing formulations of slow-release long-acting compositions of hormones are described in many of the prior arts. Such methods of preparations of slow-release long-acting hormonal compositions include the bioabsorbable fusion products of steroid and a lipoid carrier as described in U.S. Pat. No. 4,244,949 (35; Gupta G N: Manufacture of long term contraceptive implant, U.S. Pat. No. 4,244,949; 1981). Preparations of microcapsules laden with an active ingredient are described in U.S. Pat. No. 4,389,330 (33; Tice T R, and Lewis D H: Microencapsulation process, U.S. Pat. No. 4,389,330; 1983) in 1983. Similar biodegradable injectable microcapsules made of hormones and polymers such as polyortho-ester or polyacetal were used in U.S. Pat. No. 5,430,585 (34; Pike M and Spicer D V: Methods and formulations for use in treating oophorectomized women, U.S. Pat. Nos. 5,340,586; 1994) and 5,430,586. Hormonal compositions as slow-release capsules made of Silastic, Dow Corning's No 602-305 medical grade polydimethylsiloxane, an inert non-reactive tube forming polymer was used to encapsulate the hormone compositions in U.S. Pat. No. 4,210,644 (37; Ewing L L, Desjardins C: Male contraception; U.S. Pat. No. 4,210,644; 1980). As in U.S. Pat. No. 4,210,644, Silastic silicone rubber tubing is used for the preparation of levonorgestrel implant, Norplant System of Wyeth—Ayerst Laboratories as a long-acting contraceptive (43; Norplant System, Wyeth Ayerst Laboratories, Physicians Desk Reference, PDR, 51, 1997, p2868). In this invention, similar prior arts methods are adapted to prepare suitable implants of androgen suppressive formulations for the treatment of prostate cancer.

### PREFERRED EMBODIMENT—DESCRIPTION

Preparation of Biodegradable Hormonal Compositions Fused with a Lipoid Carrier for Prostatic Implants

As a preferred method of fused implant preparation for prostatic implants for hormonal treatment of prostate cancer, the methods described in U.S. Pat. No. 4,244,949 (35; Gupta G N: Manufacture of long term contraceptive implant, U.S. Pat. No. 4,244,949; 1981) more than 21 years ago is adapted. The entire disclosure of which is hereby incorporated by reference.

1. Preparation of Biodegradable Fused Prostatic Implants of DES and Cholesterol Formulation

In accordance with one preferred embodiment for one fused implant preparation of DES and cholesterol for prostatic implant, DES is purified by dissolving it in methanol, filtering through analytical grade filter paper and crystallizing it by slow addition of small amount of distilled water and allowing it to continue to crystallize slowly in a refrigerator for about 12 hours. Filtering it again through analytical grade filter paper and vacuum drying at 60° C. to a constant weight for two or more hours and storing the crystallized form of DES under nitrogen at 25° C. until it is used for fused single prostatic implant preparation. Thirty mg of purified DES and 7.5 mg of cholesterol is made to a powder form by thorough mixing under nitrogen. This mixture is then transferred into a 10 cm long, 2.4 to 2.8 mm diameter Teflon tubing and compacted with stainless steel probes from both open ends of the Teflon tubing under nitrogen. The portion of the Teflon tubing containing the DES and cholesterol mixture is heated over their melting points for 45 seconds over an aluminum block. The molten mixture is consolidated as one fused mass by pressing it with the stainless steel probes. After cooling, the probes are removed. The fused DES and cholesterol prostate implant preparation is removed from the Teflon tubing by splitting the tube walls with a blade. Based upon the need of a particular patient and on clinical testing after a test dose implant and observing the PSA level as an indicator of response to such treatment by implants, the extended period implant dose of such fused implant is adjusted to achieve the desired daily therapeutic concentration.

2. Preparation of Biodegradable Fused Prostatic Implants of Estradiol and Cholesterol Formulation

In accordance with one preferred embodiment for one fused implant preparation of estradiol and cholesterol for prostatic implant, estradiol is purified like the DES purification. And 30 mg of purified estradiol and 7.5 mg of cholesterol is fused together as per the method of fused DES and cholesterol implant preparation. This fused implant is stored under nitrogen and in aseptic conditions for future use. Based upon the need of a particular patient and on clinical testing after a test dose implant and observing the PSA levels as an indicator of response to such treatment by implants, the longer period implant dose of such fused implant is adjusted to achieve the desired daily therapeutic concentration.

3. Preparation of Iodinated Estradiol (Iodoestradiol)

Iodinated estradiol is prepared as per the methods described by this inventor in his U.S. Pat. No. 4,321,208 in 1982 with minor modifications. The high affinity binding of iodine-125 labeled estradiol to estrogen receptor sites and estrogen antiserum was shown by this inventor as early as in 1976, the filing date of patent application. At that time no other publication or information existed on iodine-125 labeled estradiol or its high affinity binding to estrogen receptor and estrogen antiserum.

In brief, non-radioactive iodoestradiol is prepared by dissolving estradiol in methanol and allowing it to react with iodine. In a preferred embodiment, sodium or potassium iodide is dissolved in water. Hydrogen peroxide or chloramine-T dissolved in small amount of water is added to free the elemental iodine from its sodium or potassium salts. Iodine reactions with estrogen molecules take place spontaneously and form the iodoestradiol. The iodinated estradiol is precipitated with water and it is separated from the reaction mixture by centrifugation.

In a preferred embodiment 8 gr. Estradiol 17-β is dissolved in 100-ml methanol and filtered through analytical filter paper. Separately, 1-gr. sodium iodide and 100 μg chloramine-T is dissolved in 5-ml water and this is added to the estradiol dissolved in methanol. The iodine labeling to estradiol takes place spontaneously. After this reaction mixture is allowed to stand for about an hour, at room temperature, about 100 ml distilled water is added slowly to precipitate the iodoestradiol. The reaction mixture is centrifuged and the sediment iodoestradiol is washed repeatedly with water to remove any residual of iodine and chloramine-T. The sediment of iodoestradiol is vacuum dried at 60° C. for two or more hours to a constant weight and it is stored under nitrogen at 25° C. until it is used.

As shown in U.S. Pat. No. 4,321,208 by this inventor, such iodinated estradiol binds to both the estrogen, the receptor sites and to estrogen antiserum indicating its similarity with the naturally occurring estradiol 17β.

4. Preparation of Biodegradable Fused Prostatic Implants of Iodoestradiol and Cholesterol Formulation

In accordance with one preferred embodiment for one fused implant preparation of iodoestradiol and cholesterol for prostatic implant. Iodoestradiol is prepared as in above description and 50 mg, of iodoestradiol and 7.5 mg of cholesterol are fused together as per the methods of fused DES and cholesterol implant preparation. Based upon the need of a particular patient and on clinical testing after a test dose implant and observing the PSA levels as an indicator of response to such treatment by implants, the implant dose of such fused implant is adjusted to achieve the desired daily therapeutic concentration.

Because of the heaviness and the electronegative characteristic of iodine in the estradiol molecule, it would render also its cytotoxic actions to the prostate cancer during this deiodination process. The highest concentration of iodoestradiol diffused daily from this iodoestradiol fused with cholesterol implant is within the prostate. Since the deiodination takes place very rapidly, the iodoestradiol's maximum deiodination associated cytotoxicity is exerted at the implant site, within the prostate. Based upon the need of a particular patient and on clinical testing after a test dose implant with the PSA levels as an indicator of response to such treatment by implants, the longer period implant dose of such fused implant is adjusted to achieve the desired daily therapeutic concentration.

5. Preparation of Biodegradable Fused Prostatic Implants of Progesterone and Cholesterol Formulation

In accordance with one preferred embodiment for one fused implant preparation of progesterone and cholesterol for the prostate, progesterone is purified like the DES purification. Thirty mg of purified progesterone and 7.5 mg of cholesterol are fused together as per the method of fused DES and cholesterol implant preparation. This fused implant is stored under nitrogen and in aseptic conditions for future use. The therapeutic dose of progesterone will inhibit the hypothalamic LHRH and pituitary LH and FSH secretions. It will suppress the androgen secretion. Furthermore, progesterone will be active in some androgen refractory prostate cancer. Based upon the need of a particular patient and on clinical testing after a test dose implant and observing the PSA levels as an indicator of response to such treatment by implants, the longer period implant dose of such fused implant is adjusted to achieve the desired daily therapeutic concentration.

6. Preparation of Biodegradable Fused Prostatic Implants of Prednisolone and Cholesterol Formulation

In accordance with one preferred embodiment for one fused implant preparation of Prednisolone and cholesterol for the prostate, prednisolone is purified like the DES purification. Thirty mg of purified prednisolone and 7.5 mg of cholesterol are fused together as per the method of fused DES and cholesterol implant preparation. This fused implant is stored under nitrogen in aseptic conditions for future use. Based upon the need of a particular patient and on clinical testing after a test dose implant and observing the PSA levels as an indicator of response to such treatment by implants, the longer period implant dose of such fused implant is adjusted to achieve the desired daily therapeutic concentration.

7. Preparation of Biodegradable Fused Prostatic Implants of Flutamide and Cholesterol Formulation

In accordance with one preferred embodiment for one fused implant preparation of flutamide and cholesterol for prostatic implant, estradiol is purified like the DES purification and 30 mg of purified estradiol and 7.5 mg of cholesterol are fused together as per the methods of fused DES and cholesterol implant preparation. This fused implant is stored under nitrogen and in aseptic conditions for future use. Based upon the need of a particular patient and on clinical testing after a test dose implant and observing the PSA levels as an indicator of response to such treatment by implants, the longer lasting implant dose of such fused implant is adjusted to achieve the desired daily therapeutic concentration.

Because these implants are within the prostate, the concentration of flutamide diffused from the flutamide fused with cholesterol is much higher in the prostate than its systemic concentration. Hence there is lesser systemic toxic effects of flutamide associated with such implants. Furthermore, since flutamide binds to androgen receptor sites competitively with testosterone, this local higher concentration of flutamide will saturate the testosterone binding sites of the prostate cancer. Hence it is a much more efficient treatment of prostate cancer than when flutamide is administered orally. Because of the high dose of orally administered flutamide, it has more systemic toxicity. Its concentration reaching the prostate by the oral administration is much lower than those achieved by its implant to the prostate. Therefore, there will not be sufficient flutamide to bind all of the testosterone receptor sites of the prostate and the prostate cancer. Hence the orally administered flutamide is less effective to inhibit the androgen dependent growth of prostate cancer. This may be the reason why presently the flutamide is thought to be less effective in the treatment of prostate cancer than it was expected. At present flutamide is administered orally.

8. Preparation of Biodegradable Fused Prostatic Implants of Estramustine and Cholesterol Formulation

In accordance with one preferred embodiment for one fused implant preparation of estramustine and cholesterol for the prostate. Estramustine is purified like the DES purification and 30 mg of purified estramustine and 7.5 mg of cholesterol are fused together as per the methods of fused DES and cholesterol implant preparation. Based upon the need of a particular patient and on clinical testing after a test dose implant and observing the PSA levels as an indicator of response to such treatment by implants, the longer period implant dose of such fused implant is adjusted to achieve the desired daily therapeutic concentration.

Because these implants are within the prostate, the concentration of estramustine diffused from the implant is much higher in the prostate than its systemic concentration. Hence there is lesser systemic toxic effects of estramustine associated with such implants. Estramustine preferentially binds to the estramustine binding protein that is abundantly present in the prostatic epithelial cells. Its estrogenic activity and its microtubular inhibitory properties mediate its cytotoxicity. The local high concentration of estramustine diffused from the implants will saturate the estramustine binding sites of the prostate cancer with high affinity. Hence it is a much more efficient treatment of prostate cancer than when estramustine is administered orally. The usually recommended dose of estramustine for the treatment of prostate cancer is 10-16 mg per kg body weight per day. Therefore, the dose for a patient weighing 70 kg would be about 1000 mg. Because of this high dose of orally administered estramustine, it has much systemic toxicity. Its concentration reaching the prostate by the oral administration is much lower than those achieved by its implant to the prostate. Therefore, there will not be sufficient estramustine to bind all of the estramustine binding protein of the prostate and the prostate cancer. Hence in spite of the high dose of the orally administered estramustine it is less effective to inhibit the tumor growth. This may be the reason why the estramustine is not as effective as it was thought to control prostate cancer.

The major metabolite of estramustine is the estrone analogue estramustine and estradiol. Like the estrogen derivative of estramustine, the estrone derivative of estramustine will bind to its binding proteins in the prostatic epithelial cells. Because these implants are within the prostate, high concentration estramustine will be diffused from the capsules to the prostate than when it is given orally. Since the high affinity prostatic epithelial cell bound estramustine is metabolized to estrogen, such estrogen will also saturate the estrogen binding sites of prostate cancer which will exert the beneficial actions of estrogen on prostate cancer with lesser toxicity of otherwise systemically given estrogen.

9. Preparation of Biodegradable Fused Prostatic Implants of DES, Prednisolone and Cholesterol Formulation

In accordance with one preferred embodiment for one fused combination implant preparation of DES, prednisolone and cholesterol for prostatic implant, DES and prednisolone are purified as in DES purification. Thirty mg of purified DES and 30 mg of prednisolone and 15 mg of cholesterol are mixed and fused together as per the methods of fused DES and cholesterol implant preparation. This fused implant is stored under nitrogen and in aseptic conditions for future use. Based upon the need of a particular patient and on clinical testing after a test dose implant and observing the PSA levels as an indicator of response to such treatment by implants, the extended period implant dose of such fused implant is adjusted to achieve the desired daily therapeutic concentration. Because these implants are within the prostate, the concentration of DES and prednisolone released from such implant is much higher in the prostate than their systemic concentration and hence this formulation exerts its maximum therapeutic effects in the prostate with no significant systemic toxicity. Furthermore, this higher concentration of this formulation in the prostate will saturate the estrogen and prednisolone binding sites of prostate cancer. This formulation is also active in some androgen refractory prostate cancer.

10. Preparation of Biodegradable Fused Prostatic Implants of DES, Flutamide and Cholesterol Formulation

In accordance with one preferred embodiment for one fused combination implant preparation of DES, Flutamide and cholesterol, the DES and flutamide are purified as in the DES purification. Thirty mg of purified DES, 30 mg of flutamide and 15 mg of cholesterol are mixed and fused together as per the method of fused DES and cholesterol implant preparation. This fused implant is stored under nitrogen and in aseptic conditions for future use. Based upon the need of a particular patient and on clinical testing after a test dose implant and observing the PSA levels as an indicator of response to such treatment by implants, the extended period implant dose of such fused implant is adjusted to achieve the desired daily therapeutic concentration. Because these implants are within the prostate, the concentration of DES and flutamide released from such implant is much higher in the prostate than their systemic concentration and hence this formulation exerts its maximum therapeutic effects in the prostate with no significant systemic toxicity. Furthermore, this higher concentration of this formulation in the prostate will saturate the estrogen and progesterone binding sites of prostate cancer. This is also active as androgen refractory prostate cancer.

11. Preparation of Biodegradable Fused Prostatic Implants of DES, Progesterone and Cholesterol Formulation

In accordance with one preferred embodiment for one fused implant preparation of DES, progesterone and cholesterol for the prostate, DES and progesterone are purified as in the DES purification. Thirty mg of purified DES and 30 mg of progesterone and 15 mg of cholesterol are fused together as per the method of fused DES and cholesterol implant preparation. This fused implant is stored under nitrogen and in aseptic conditions for future use. Based upon the need of a particular patient and on clinical testing after a test dose implant and observing the PSA levels as an indicator of response to such treatment by implants, the longer period implant dose of such fused implant is adjusted to achieve the desired daily therapeutic concentration. Because these implants are within the prostate, the concentrations of DES and progesterone released from such implant is much higher in the prostate than their systemic concentrations and hence this formulation exerts its maximum therapeutic effects in the prostate with no significant systemic toxicity. Furthermore, this higher concentration of this formulation in the prostate will saturate the estrogen and progesterone binding sites of prostate cancer. This is also active as androgen refractory prostate cancer.

12. Preparation of Biodegradable Prostatic Implant of Fused of Estradiol, Prednisolone and Cholesterol Formulation

In accordance with one preferred embodiment for one fused combination implant preparation of estradiol, prednisolone and cholesterol for the prostate. Estradiol and prednisolone are purified like the DES purification. Thirty mg of purified estradiol and 30 mg of prednisolone and 15 mg of cholesterol are mixed and fused together as per the methods of fused DES and cholesterol implant preparation. This fused implant is stored under nitrogen and in aseptic conditions for future use. Based upon the need of a particular patient and on clinical testing after a test dose implant and observing the PSA levels as an indicator of response to such treatment by implants, the extended period implant dose of such fused implant is adjusted to achieve the desired daily therapeutic concentration. Because these implants are within the prostate, the concentration of estrogen and prednisolone released from such implant is much higher in the prostate than their systemic concentration and hence this formulation exerts its maximum therapeutic effects in the prostate with no significant systemic toxicity. Furthermore, this higher concentration of formulation in the prostate will saturate the estrogen and prednisolone binding sites of prostate cancer. This estrogen and prednisolone formulation is also active in some androgen refractory prostate cancer.

13. Preparation of Biodegradable Fused Prostatic Implants of Estradiol, Progesterone and Cholesterol Formulation

In accordance with one preferred embodiment for one fused implant preparation of estradiol progesterone and cholesterol for the prostate. Estradiol and progesterone are purified as in the DES purification method. Thirty mg of purified estradiol, 30 mg of progesterone and 15 mg of cholesterol are fused together as per the methods of fused DES and cholesterol implant. This fused implant is stored under nitrogen and in aseptic conditions for future use. Based upon the need of a particular patient and on clinical testing after a test dose implant and observing the PSA levels as an indicator of response to such implant treatment, the extended period implant dose of such fused implant is adjusted to achieve the desired daily therapeutic concentration. Because these implants are within the prostate, the concentrations of estradiol and progesterone released from such implant is much higher in the prostate than their systemic concentration and hence this formulation exerts its maximum therapeutic effects in the prostate with no significant systemic toxicity. Furthermore, this higher concentration of this formulation in the prostate will saturate the estrogen and progesterone binding sites of prostate cancer. This estradiol and progesterone formulation is also active in some androgen refractory prostate cancer.

14. Preparation of Biodegradable Fused Prostatic Implants of Estradiol, Flutamide and Cholesterol Formulation

In accordance with one preferred embodiment for one fused combination implant preparation of estradiol, flutamide and cholesterol for the prostate Estradiol and flutamide are purified like the DES purification method. Thirty mg of purified estradiol and thirty mg of flutamide and 15 mg of cholesterol are mixed and fused together as per the methods of fused DES and cholesterol implant preparation. This fused implant is stored under nitrogen and in aseptic conditions for future use. Based upon the need of a particular patient and on clinical testing after a test dose implant and observing the PSA levels as an indicator of response to such treatment by implants, the extended period implant dose of such fused implant is adjusted to achieve the desired daily therapeutic concentration. Because these implants are within the prostate, the concentrations of estrogen and flutamide released from such implant is much higher in the prostate than their systemic concentrations and hence this formulation exerts its maximum therapeutic effects in the prostate with no significant systemic toxicity. Furthermore, this higher concentration of this formulation in the prostate will saturate the estrogen and androgen binding sites of prostate cancer. This estrogen and flutamide formulation is also active in some androgen refractory prostate cancer.

15. Preparation of Biodegradable Fused Prostatic Implants of Estramustine, Prednisolone and Cholesterol Formulation

In accordance with one preferred embodiment for one fused combination implant preparation of estramustine, prednisolone and cholesterol for prostatic implant. Estramustine and prednisolone are purified like the DES purification and thirty mg of purified estramustine and thirty mg of prednisolone and 15 mg of cholesterol are mixed and fused together as per the methods of fused DES and cholesterol implant preparation. This fused implant is stored under nitrogen and in aseptic conditions for future use. Based upon the need of a particular patient and on clinical testing after a test dose implant and observing the PSA levels as an indicator of response to such treatment by implants, the extended period implant dose of such fused implant is adjusted to achieve the desired daily therapeutic concentration. Because these implants are within the prostate, the concentrations of estramustine and prednisolone released from such implant is much higher in the prostate than their systemic concentrations and hence this formulation exerts its maximum therapeutic effects in the prostate with no significant systemic toxicity. Furthermore, this higher concentration of this formulation in the prostate will saturate the estrogen and prednisolone binding sites of prostate cancer. This estramustine and prednisolone formulation is also active in some androgen refractory prostate cancer.

16. Preparation of Biodegradable Fused Prostatic Implants of Estramustine, Flutamide and Cholesterol Formulation

In accordance with one preferred embodiment for one fused combination implant preparation of estramustine, flutamide and cholesterol for prostatic implant, estramustine and flutamide are purified like the DES purification. Thirty mg of purified estramustine and thirty mg of flutamide and 15 mg of cholesterol are mixed and fused together as per the methods of fused DES and cholesterol implant preparation. This fused implant is stored under nitrogen and in aseptic conditions for future use. Based upon the need of a particular patient and on clinical testing after a test dose implant and observing the PSA levels as an indicator of response to such treatment by implants, the extended period implant dose of such fused implant is adjusted to achieve the desired daily therapeutic concentration. Because these implants are within the prostate, the concentrations of estramustine and flutamide released from such implant is much higher in the prostate than their systemic concentrations and hence this formulation exerts its maximum therapeutic effects in the prostate with no significant systemic toxicity. Furthermore, this higher concentration of this formulation in the prostate will saturate the estrogen and androgen binding sites of prostate cancer. This estramustine and flutamide formulation is also active in some androgen refractory prostate cancer.

17. Preparation of Biodegradable Fused Prostatic Implants of Estramustine, Progesterone and Cholesterol Formulation

In accordance with one preferred embodiment for one fused combination implant preparation of estramustine, progesterone and cholesterol for prostatic implant, estramustine and progesterone are purified like the DES purification. Thirty mg of purified estramustine and thirty mg of progesterone and 15 mg of cholesterol are mixed and fused together as per the methods of fused DES and cholesterol implant preparation. This fused implant is stored under nitrogen and in aseptic conditions for future use. Based upon the need of a particular patient and on clinical testing after a test dose implant and observing the PSA levels as an indicator of response to such treatment by implants, the extended period implant dose of such fused implant is adjusted to achieve the desired daily therapeutic concentration. Because of these implants being within the prostate, the concentrations of estramustine and progesterone released from such implant is much higher in the prostate than their systemic concentrations and hence this formulation exerts its maximum therapeutic effects in the prostate with no significant systemic toxicity.

Furthermore, this higher concentration of this formulation in the prostate will saturate the estrogen and progesterone binding sites of prostate cancer. This estramustine and progesterone formulation is active in some androgen refractory prostate cancer.

18. Preparation of Biodegradable Fused Prostatic Implants of DES, Prednisolone, Flutamide and Cholesterol Formulation

In accordance with one preferred embodiment for one fused combination implant preparation of DES, prednisolone, flutamide and cholesterol for prostatic implant, DES, prednisolone and flutamide are purified like the DES purification. Thirty mg of purified DES, thirty mg of prednisolone and thirty mg of flutamide and 22.5 mg of cholesterol are mixed and fused together as per the methods of fused DES and cholesterol implant preparation. This fused implant is stored under nitrogen and in aseptic conditions for future use. Based upon the need of a particular patient and on clinical testing after a test dose implant and observing the PSA levels as an indicator of response to such treatment by implants, the extended period implant dose of such fused implant is adjusted to achieve the desired daily therapeutic concentration. Because of these implants being within the prostate, the concentrations of DES, prednisolone and flutamide released from such implant is much higher in the prostate than their systemic concentrations and hence this formulation exerts its maximum therapeutic effects in the prostate with no significant systemic toxicity. Furthermore, this higher concentration of this formulation in the prostate will saturate the estrogen, prednisolone and androgen binding sites of prostate cancer. This DES, prednisolone and flutamide formulation is also active in some androgen refractory prostate cancer.

19. Preparation of Biodegradable Fused Prostatic Implants of DES, Progesterone, Flutamide and Cholesterol Formulation

In accordance with one preferred embodiment for one fused combination implant preparation of DES, progesterone, flutamide and cholesterol for prostatic implant, DES, progesterone and flutamide are purified like the DES purification. Thirty mg of purified DES, thirty mg of progesterone and thirty mg of flutamide and 22.5 mg of cholesterol are mixed and fused together as per the methods of fused DES and cholesterol implant preparation. This fused implant is stored under nitrogen and in aseptic conditions for future use. Based upon the need of a particular patient and on clinical testing after a test dose implant and observing the PSA levels as an indicator of response to such treatment by implants, the extended period implant dose of such fused implant is adjusted to achieve the desired daily therapeutic concentration. Because of these implants being within the prostate, the concentrations of DES, progesterone and flutamide released from such implant is much higher in the prostate than their systemic concentrations and hence this formulation exerts its maximum therapeutic effects in the prostate with no significant systemic toxicity. Furthermore, this higher concentration of this formulation in the prostate will saturate the estrogen, progesterone and androgen binding sites of prostate cancer. This DES, progesterone and flutamide combination will also be active in some androgen refractory prostate cancer.

20. Preparation of Biodegradable Fused Prostatic Implants of Estradiol, Prednisolone, Flutamide and Cholesterol Formulation

In accordance with one preferred embodiment for one fused combination implant preparation of estradiol, prednisolone, flutamide and cholesterol for prostatic implant, estradiol, prednisolone and flutamide are purified like the DES purification. Thirty mg of purified estradiol, thirty mg of prednisolone and thirty mg of flutamide and 22.5 mg of cholesterol are mixed and fused together as per the methods of fused DES and cholesterol implant preparation. This fused implant is stored under nitrogen and in aseptic conditions for future use. Based upon the need of a particular patient and on clinical testing after a test dose implant and observing the PSA levels as an indicator of response to such treatment by implants, the extended period implant dose of such fused implant is adjusted to achieve the desired daily therapeutic concentration. Because these implants are within the prostate, the concentrations of estradiol, prednisolone and flutamide released from such implant is much higher in the prostate than their systemic concentrations and hence this formulation exerts its maximum therapeutic effects in the prostate with no significant systemic toxicity. Furthermore, this higher concentration of this formulation in the prostate will saturate the estrogen, prednisolone and androgen binding sites of prostate cancer. This formulation is also active in some androgen refractory prostate cancer.

21. Preparation of Biodegradable Fused Prostatic Implants of Estradiol, Progesterone, Flutamide and Cholesterol Formulation

In accordance with one preferred embodiment for one fused combination implant preparation of estradiol, progesterone, flutamide and cholesterol for prostatic implant, estradiol, progesterone and flutamide are purified like the DES purification. Thirty mg of purified estradiol, thirty mg of progesterone and thirty mg of flutamide and 22.5 mg of cholesterol are mixed and fused together as per the methods of fused DES and cholesterol implant preparation. This fused implant is stored under nitrogen and in aseptic conditions for future use. Based upon the need of a particular patient and on clinical testing after a test dose implant and observing the PSA levels as an indicator of response to such treatment by implants, the extended period implant dose of such fused implant is adjusted to achieve the desired daily therapeutic concentration. Because these implants are within the prostate, the concentrations of estradiol, progesterone and flutamide released from such implant is much higher in the prostate than their systemic concentrations and hence this formulation exerts its maximum therapeutic effects in the prostate with no significant systemic toxicity. Furthermore, this local higher concentration of this formulation will saturate the estrogen, progesterone and androgen binding sites of prostate cancer. This estradiol, progesterone and flutamide formulation will also be active in some androgen refractory prostate cancer.

Preparation of Slow-Release Hormonal Compositions in Silastic Capsules for Prostatic Implants

As a preferred method of slow-release Hormonal Compositions in Silastic Capsules for prostatic implants for hormonal treatment of prostate cancer, the methods described in U.S. Pat. No. 4,210,644 (37; Ewing L L, Desjardins C: Male contraception; U.S. Pat. No. 4,210,644; 1980) more than 21 years ago is adapted. The entire disclosure of which is hereby incorporated by reference. Similar encapsulated levonorgestrel implant, Norplant System of Wyeth—Ayerst Laboratories is used as a long-acting contraceptive (43; Norplant System, Wyeth Ayerst Laboratories, Physicians Desk Reference, PDR, 51, 1997, p2868). Similarly, any of the many previously known prior art methods for the preparation of microencapsulated compositions could be used for the preparation of microencapsulated steroid hormones and their synthetic derivatives as prostatic implants for the treatment and prevention of prostate cancer of this invention.

1. Preparation of Silastic Slow-Release Capsules Containing DES for Prostatic Implant

In accordance with one preferred embodiment for preparation of Silastic slow release capsules containing DES for prostatic implant, the following method is adapted. The Dow Corning Silastic, dimethylsyloxane/methylvinyalsiloxane copolymer, tubing of 0.2-mm wall thickness and 2.4 to 3.18 mm outer diameters and of 3.5 cm in length is cut. One end is closed with Silastic adhesive (polydimethylsiloxane). DES is filled into the cut tube through the open end at a dose of 30 mg. After the filling with DES, the open end of the tube is also closed with Silastic adhesive.

Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because these implants are within the prostate, the concentration of DES diffused from the capsules is much higher in the prostate than its systemic concentration and hence DES exerts its maximum therapeutic effects in the prostate with no significant systemic toxicity. Furthermore, this local higher concentration of DES will saturate the estrogen binding sites of prostate cancer.

2. Preparation of Silastic Slow-Release Capsules Containing Estradiol for Prostatic Implant

In accordance with one preferred embodiment for preparation of Silastic slow release capsules containing estradiol for prostatic implant, the following method is adapted. The Dow Corning Silastic, dimethylsyloxane/methylvinyalsiloxane copolymer, tubing of 0.2-mm wall thickness and 2.4 to 3.18 mm outer diameters and of 3.5 cm in length is cut. One end is closed with Silastic adhesive (polydimethylsiloxane). Estradiol is filled into the cut tube through the open end at a dose of 30 mg. After the filling with estradiol, the open end of the tube is also closed with Silastic adhesive.

Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity. Furthermore, this local higher concentration of estradiol will saturate the estrogen binding sites of prostate cancer.

3. Preparation of Silastic Slow-Release Capsules Containing Iodinated Estradiol for Prostatic Implant

In accordance with one preferred embodiment for preparation of Silastic slow release capsules containing iodoestradiol for prostatic implant, the following method is adapted. The Dow Corning Silastic, dimethylsyloxane/methylvinyalsiloxane copolymer, tubing of 0.2-mm wall thickness and 2.4 to 3.18 mm outer diameters and of 3.5 cm in length is cut. One end is closed with Silastic adhesive (polydimethylsiloxane). Iodoestradiol is filled into the cut tube through the open end at a dose of 30 mg. After the filling with iodoestradiol, the open of the tube end is also closed with Silastic adhesive.

Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity. Furthermore, this local higher concentration of iodinated estradiol will saturate the estrogen binding sites of prostate cancer.

4. Preparation of Silastic Slow-Release Capsules Containing Progesterone for Prostatic Implant

In accordance with one preferred embodiment for preparation of Silastic slow release capsules containing progesterone for prostatic implant, the following method is adapted. The Dow Corning Silastic, dimethylsyloxane/methylvinyalsiloxane copolymer, tubing of 0.2-mm wall thickness and 2.4 to 3.18 mm outer diameters and of 3.5 cm in length is cut. One end is closed with Silastic adhesive (polydimethylsiloxane). Progesterone is filled into the cut tube through the open end at a dose of 30 mg. After the filling with estradiol, the open end of the tube is also closed with Silastic adhesive.

Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because of these implants being within the prostate, the concentration of its contents diffused from the capsules is much higher in the prostate than its systemic concentration and hence it exerts its maximum therapeutic effects in the prostate with no significant systemic toxicity.

5. Preparation of Silastic Slow-Release Capsules Containing Prednisolone for Prostatic Implant

In accordance with one preferred embodiment for preparation of Silastic slow release capsules containing prednisolone for prostatic implant, the following method is adapted. The Dow Corning Silastic, dimethylsyloxane/methylvinyalsiloxane copolymer, tubing of 0.2-mm wall thickness and 2.4 to 3.18 mm. outer diameters and 3.5 cm in length is cut. One end is closed with Silastic adhesive (polydimethylsiloxane). Prednisolone is filled into the cut tube through the open end at a dose of 30 mg. After the filling with prednisolone, the open end of the tube is also closed with Silastic adhesive.

Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because of these implants being within the prostate, the concentration of its contents diffused from the capsules is much higher in the prostate than its systemic concentration and hence it exerts its maximum therapeutic effects in the prostate with no significant systemic toxicity. Prednisolone suppresses the adrenal synthesis of androgens including the adrenal testosterone. It is also very effective in hormone refractory prostate cancer.

6. Preparation of Silastic Slow-Release Capsules Containing Flutamide for Prostatic Implant

In accordance with one preferred embodiment for preparation of Silastic slow release capsules containing flutamide for prostatic implant, the following method is adapted. The Dow Corning Silastic, dimethylsyloxane/methylvinyalsiloxane copolymer, tubing of 0.2-mm wall thickness and 2.4 to 3.18 mm. outer diameters and 3.5 cm in length is cut. One end is closed with Silastic adhesive (polydimethylsiloxane). Flutamide is filled into the cut tube through the open end at a dose of 30 mg. After the filling with flutamide, the open end of the tube is also closed with Silastic adhesive.

Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity. Flutamide binds to androgen receptor sites competitively with testosterone, this local higher concentration of flutamide will saturate the testosterone binding sites of prostate cancer.

7. Preparation of Silastic Slow-Release Capsules Containing Estramustine for Prostatic Implant

In accordance with one preferred embodiment for preparation of Silastic slow release capsules containing estramustine for prostatic implant, the following method is adapted. The Dow Corning Silastic, dimethylsyloxane/methylvinyalsiloxane copolymer, tubing of 0.2-mm wall thickness and 2.4 to 3.18 mm outer diameters and of 3.5 cm in length is cut and its one end is closed with Silastic adhesive (polydimethylsiloxane). Estramustine is filled into the cut tube through the open end at a dose of 30 mg. After the filling with estramustine, the open end of the tube is also closed with Silastic adhesive.

Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because of these implants being within the prostate, the concentration of its contents diffused from the capsules is much higher in the prostate than its systemic concentration and hence it exerts its maximum therapeutic effects in the prostate with no significant systemic toxicity. Estramustine preferentially binds to the estramustine binding protein that is abundantly present in the prostatic epithelial cells. Its estrogenic activity and its microtubular inhibitory properties mediate its cytotoxicity. The major metabolite of estramustine is the estrone analogue estramustine and estradiol. Like the estrogen derivative of estramustine, the estrone derivative of estramustine will bind to its binding proteins in the prostatic epithelial cells. It thus enhances the beneficial actions of estrogen on prostate cancer.

8. Preparation of Silastic Slow-Release Capsules Containing DES and Prednisolone for Prostatic Implant.

In accordance with one preferred embodiment for preparation of Silastic slow release capsules containing DES and prednisolone for prostatic implant, the following method is adapted. The Dow Corning Silastic, dimethylsyloxane/methylvinyalsiloxane copolymer, tubing of 0.2-mm wall thickness and 2.4 to 3.18 mm. outer diameters and 3.5 cm in length is cut. One end is closed with Silastic adhesive (polydimethylsiloxane). DES and prednisolone are filled into the cut tube through the open end at a dose of 30 mg each. After the filling with DES and prednisolone, the open end of the tube is also closed with Silastic adhesive.

Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity. Prednisolone also suppresses the adrenal synthesis of androgens including the adrenal testosterone synthesis. Furthermore, this local higher concentration of DES will saturate the estrogen binding sites of prostate cancer.

This combination implants of DES and prednisolone enhances the tumor control by their combined cytotoxicity and androgen suppressive actions. Such a combination is effective in both hormone dependent and hormone refractory prostate cancer.

9. Preparation of Silastic Slow-Release Capsules Containing DES and Flutamide for Prostatic Implant.

In accordance with one preferred embodiment for preparation of Silastic slow release capsules containing DES and flutamide for prostatic implant, the following method is adapted. The Dow Corning Silastic, dimethylsyloxane/methylvinyalsiloxane copolymer, tubing of 0.2-mm wall thickness and 2.4 to 3.18 mm outer diameters and of 3.5 cm in length is cut and its one end is closed with Silastic adhesive (polydimethylsiloxane). DES and flutamide are filled into the cut tube through the open end at a dose of 30 mg each. After the filling with DES and flutamide, the open end of the tube is also closed with Silastic adhesive.

Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because of these implants being within the prostate, the concentration of its contents diffused from the capsules is much higher in the prostate than its systemic concentration and hence it exerts its maximum therapeutic effects in the prostate with no significant systemic toxicity. This local higher concentration of DES and flutamide will saturate both the estrogen and the androgen binding sites of prostate cancer. Their combined cytotoxicity and androgen suppressive actions are effective in both hormone dependent and hormone refractory prostate cancer.

10. Preparation of Silastic Slow-Release Capsules Containing DES and Progesterone for Prostatic Implant.

In accordance with one preferred embodiment for preparation of Silastic slow release capsules containing DES and progesterone for prostatic implant, the following method is adapted. The Dow Corning Silastic, dimethylsyloxane/methylvinyalsiloxane copolymer, tubing of 0.2-mm wall thickness and 2.4 to 3.18 mm. outer diameters and 3.5 cm in length is cut. One end is closed with Silastic adhesive (polydimethylsiloxane). DES and progesterone are filled into the cut tube through the open end at a dose of 30 mg each. After the filling with DES and progesterone, the open end of the tube is also closed with Silastic adhesive.

Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity. The local higher concentration of DES and progesterone will saturate both the estrogen and progesterone binding sites of prostate cancer. It enhances the tumor control by their combined cytotoxicity and androgen suppressive actions. Such a combination is effective in both hormone dependent and hormone refractory prostate cancer.

11. Preparation of Silastic Slow-Release Capsules Containing Estradiol and Prednisolone for Prostatic Implant.

In accordance with one preferred embodiment for preparation of Silastic slow release capsules containing estradiol and prednisolone for prostatic implant, the following method is adapted. The Dow Corning Silastic, dimethylsyloxane/methylvinyalsiloxane copolymer, tubing of 0.2-mm wall thickness and 2.4 to 3.18 mm outer diameters and 3.5 cm in length is cut. One end is closed with Silastic adhesive (polydimethylsiloxane). Estradiol and prednisolone are filled into the cut tube through the open end at a dose of 30 mg each. After the filling with estradiol and prednisolone, the open end of the tube is also closed with Silastic adhesive.

Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity. Prednisolone also suppresses the adrenal synthesis of androgens including the adrenal testosterone synthesis. Furthermore, this local higher concentration of estradiol will saturate the estrogen binding sites of prostate cancer. This combination implants of estradiol and prednisolone enhances the tumor control by their combined cytotoxicity and androgen suppressive actions. Such a combination is effective in both hormone dependent and hormone refractory prostate cancer.

12. Preparation of Silastic Slow-Release Capsules Containing Estradiol and Progesterone for Prostatic Implant.

In accordance with one preferred embodiment for preparation of Silastic slow release capsules containing estradiol and progesterone for prostatic implant, the following method is adapted. The Dow Corning Silastic, dimethylsyloxane/methylvinyalsiloxane copolymer, tubing of 0.2-mm wall thickness and 2.4 to 3.18 mm. outer diameters and 3.5 cm in length is cut. One end is closed with Silastic adhesive (polydimethylsiloxane). Estradiol and progesterone are filled into the cut tube through the open end at a dose of 30 mg each. After the filling with estradiol and progesterone, the open end of the tube is also closed with Silastic adhesive.

Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity. The local higher concentration of estradiol will saturate the estrogen binding sites of the prostate cancer. This combination implants of estradiol and progesterone enhances the tumor control by their combined cytotoxicity and androgen suppressive actions. Such a combination is effective in both hormone dependent and hormone refractory prostate cancer.

13. Preparation of Silastic Slow-Release Capsules Containing Estradiol and Flutamide for Prostatic Implant.

In accordance with one preferred embodiment for preparation of Silastic slow release capsules containing estradiol and flutamide for prostatic implant, the following method is adapted. The Dow Corning Silastic, dimethylsyloxane/methylvinyalsiloxane copolymer, tubing of 0.2-mm wall thickness and 2.4 to 3.18 mm outer diameters and of 3.5 cm in length is cut and its one end is closed with Silastic adhesive (polydimethylsiloxane). Estradiol and flutamide are filled into the cut tube through the open end at a dose of 30 mg each. After the filling with estradiol and flutamide, the open end of the tubing is also closed with Silastic adhesive.

Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because of these implants being within the prostate, the concentration of its contents diffused from the capsules is much higher in the prostate than its systemic concentration and hence it exerts its maximum therapeutic effects in the prostate with no significant systemic toxicity.

The local higher concentration of estradiol will saturate the estrogen binding sites of prostate cancer. Furthermore, since flutamide binds to androgen receptor sites competitively with testosterone, this local higher concentration of flutamide will saturate the testosterone binding sites of prostate cancer. Hence such a combination of estradiol and flutamide is a much more efficient treatment of prostate cancer than when flutamide is administered orally. Their concentrations reaching the prostate by the oral administration is much lower than those achieved by their prostatic implant. Hence there will not be sufficient estrogen and flutamide to bind all of the estrogen and testosterone receptor sites of the prostate and the prostate cancer. Hence the orally administered estradiol and flutamide is less effective to inhibit the androgen dependent growth of prostate cancer. At present flutamide is administered orally. This combination implants of estradiol and flutamide enhances the tumor control by their combined cytotoxicity and androgen suppressive actions. Such a combination is effective in both hormone dependent and hormone refractory prostate cancer.

14. Preparation of Silastic Slow-Release Capsules Containing Estramustine and Prednisolone for Prostatic Implant

In accordance with one preferred embodiment for preparation of Silastic slow release capsules containing estramustine and prednisolone for prostatic implant, the following method is adapted. The Dow Corning Silastic, dimethylsyloxane/methylvinyalsiloxane copolymer, tubing of 0.2-mm wall thickness and 2.4 to 3.18 mm outer diameters and of 3.5 cm in length is cut and its one end is closed with Silastic adhesive (polydimethylsiloxane). Estramustine and prednisolone are filled into the cut tube through the open end at a dose of 30 mg each. After the filling with estramustine and prednisone, the open end of the tube is also closed with Silastic adhesive. Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Estramustine preferentially binds to the estramustine binding protein that is abundantly present in the prostatic epithelial cells. Its estrogenic activity and its microtubular inhibitory properties mediate its cytotoxicity. The local high concentration of estramustine diffused from the implanted capsules will saturate the estramustine binding sites of the prostate cancer. Hence it is a much more efficient treatment of prostate cancer than when estramustine is administered orally. The usually recommended dose of estramustine for the treatment of prostate cancer is 10-16 mg per kg body weight per day. Therefore, the dose for a patient weighing 70 kg would be about 1000 mg. Because of this high oral dose, it has much systemic toxicity. Its concentration reaching the prostate by the oral administration is much lower than those achieved by its implant to the prostate. Therefore, there will not be sufficient estramustine to bind all of the estramustine binding protein of the prostate and the prostate cancer. Hence in spite of the high dose of the orally administered estramustine it is less effective to inhibit the tumor growth. This may be the reason why the estramustine is not as effective as it was thought to be to control the prostate cancer. The major metabolites of estramustine are its estrogen and estrone analogue estramustine. Like the estrogen derivative of estramustine, the estrone derivative of estramustine binds to its binding proteins in the prostatic epithelial cells. Since the high affinity prostatic epithelial cell bound estramustine is metabolized to estrogen and since there will be abundant such metabolized estrogen within the cells, it will saturate the cell's capacity to bind estrogen to its estrogen binding sites. It thus enhances the beneficial actions of estrogen on prostate cancer. Prednisolone suppresses the adrenal synthesis of androgens including the adrenal testosterone. It is also very effective in hormone refractory prostate cancer. This combination implants of estramustine and prednisolone enhances tumor control by their combined cytotoxicity and androgen suppressive actions. Such a combination is effective in both hormone dependent and hormone refractory prostate cancer.

15. Preparation of Silastic Slow-Release Capsules Containing Estramustine and Flutamide for Prostatic Implant

In accordance with one preferred embodiment for preparation of Silastic slow release capsules containing estramustine and flutamide for prostatic implant, the following method is adapted. The Dow Corning Silastic, dimethylsyloxane/methylvinyalsiloxane copolymer, tubing of 0.2-mm. wall thickness and 2.4 to 3.18 mm. outer diameters and 3.5 cm in length is cut. One end is closed with Silastic adhesive (polydimethylsiloxane). Estramustine and flutamide are filled into the cut tube through the open end at a dose of 30 mg each. After the filling with estramustine and flutamide, the open end of the tube is also closed with Silastic adhesive.

Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity. This combination of estramustine and flutamide enhances the tumor control by their combined cytotoxicity and androgen suppressive actions. Such combination is effective in both hormone dependent and hormone refractory prostate cancer.

16. Preparation of Silastic Slow-Release Capsules Containing Estramustine and Progesterone for Prostatic Implant

In accordance with one preferred embodiment for preparation of Silastic slow release capsules containing estramustine and progesterone for prostatic implant, the following method is adapted. The Dow Corning Silastic, dimethylsyloxane/methylvinyalsiloxane copolymer, tubing of 0.2-mm wall thickness and 2.4 to 3.18 mm outer diameters and 3.5 cm in length is cut. One end is closed with Silastic adhesive (polydimethylsiloxane). Estramustine and progesterone are filled into the cut tube through the open end at a dose of 30 mg each. After the filling with estramustine and progesterone, the open end of the tube is also closed with Silastic adhesive.

Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity. This combination of estramustine and progesterone enhances the tumor control by their combined cytotoxicity and androgen suppressive actions. Such a formulation is effective in both hormone dependent and hormone refractory prostate cancer.

17. Preparation of Silastic Slow-Release Capsules Containing DES, Prednisolone and Flutamide for Prostatic Implant

In accordance with one preferred embodiment for preparation of Silastic slow release capsules containing DES, prednisolone and flutamide for prostatic implant, the following method is adapted. The Dow Corning Silastic, dimethylsyloxane/methylvinyalsiloxane copolymer, tubing of 0.2-mm wall thickness and 2.4 to 3.18 mm outer diameters and 4 cm in length is cut. One end is closed with Silastic adhesive (polydimethylsiloxane). DES, prednisolone and flutamide are filled into the cut tube through the open end at a dose of 30 mg each. After the filling with DES, prednisolone and flutamide, the open end of the tube is also closed with Silastic adhesive.

Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity. This triple drug formulation of DES, prednisolone and flutamide enhances the prostatic tumor control by their combined cytotoxicity and androgen suppressive actions. Such combination is effective in both hormone dependent and hormone refractory prostate cancer.

18. Preparation of Silastic Slow-Release Capsules Containing DES, Progesterone and Flutamide for Prostatic Implant

In accordance with one preferred embodiment for preparation of Silastic slow release capsules containing DES, progesterone and flutamide for prostatic implant, the following method is adapted. The Dow Corning Silastic, dimethylsyloxane/methylvinyalsiloxane copolymer, tubing of 0.2-mm wall thickness and 2.4 to 3.18 mm outer diameters and of 4 cm in length is cut and its one end is closed with Silastic adhesive (polydimethylsiloxane). DES, progesterone and flutamide are filled into the cut tube through the open end at a dose of 30 mg each. After the filling with DES, progesterone and flutamide, the open end of the tube is also closed with Silastic adhesive.

Based upon the need of a particular patient and on clinical testing after a test dose implant and observing the PSA levels as an indicator of response to such implant treatment, the longer period implant dose of such encapsulated implant is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity. This triple drug combination of DES, progesterone and flutamide enhances the prostatic tumor control by their combined cytotoxicity and androgen suppressive actions. Such combination is effective in both hormone dependent and hormone refractory prostate cancer.

19. Preparation of Silastic Slow-Release Capsules Containing Estradiol, Prednisolone and Flutamide for Prostatic Implant

In accordance with one preferred embodiment for preparation of Silastic slow release capsules containing estradiol, prednisolone and flutamide for prostatic implant, the following method is adapted. The Dow Corning Silastic, dimethylsyloxane/methylvinyalsiloxane copolymer, tubing of 0.2-mm wall thickness and 2.4 to 3.18 mm outer diameters and of 4 cm in length is cut and its one end is closed with Silastic adhesive (polydimethylsiloxane). Estradiol, prednisolone and flutamide are filled into the cut tube through the open end at a dose of 30 mg each. After the filling with estradiol, prednisolone and flutamide, the open end of the tube is also closed with Silastic adhesive.

Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity. This triple drug formulation of estradiol, prednisolone and flutamide enhances the prostatic tumor control by their combined cytotoxicity and androgen suppressive actions. Such combination is effective in both hormone dependent and hormone refractory prostate cancer.

20. Preparation of Silastic Slow-Release Capsules Containing Estradiol, Progesterone and Flutamide for Prostatic Implant

In accordance with one preferred embodiment for preparation of Silastic slow release capsules containing estradiol, progesterone and flutamide for prostatic implant, the following method is adapted. The Dow Corning Silastic, dimethylsyloxane/methylvinyalsiloxane copolymer, tubing of 0.2-mm wall thickness and 2.4 to 3.18 mm outer diameters and of 4 cm in length is cut and its one end is closed with Silastic adhesive (polydimethylsiloxane). Estradiol, progesterone and flutamide are filled into the cut tube through the open end at a dose of 30 mg each. After the filling with estradiol, progesterone and flutamide, the open end of the tube is also closed with Silastic adhesive.

Based upon the need of a particular patient and on clinical testing after a test dose implant and observing the PSA levels as an indicator of response to such implant treatment, the extended period implant dose of such encapsulated implant is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity. This triple drug combination of estradiol, progesterone and flutamide enhances the prostatic tumor control by their combined cytotoxicity and androgen suppressive actions. Such combination is effective in both hormone dependent and hormone refractory prostate cancer.

Preparation of Slow-Release Hormonal Compositions in Microcapsules for Prostatic Implants

As a preferred method of slow-release hormonal compositions in microcapsules for the treatment of prostate cancer as prostatic implants, the methods described in U.S. Pat. No. 4,389,330 (33; Tice T R, and Lewis D H: Microencapsulation process, U.S. Pat. No. 4,389,330; 1983) more than 18 years ago is adapted. The entire disclosure of which is hereby incorporated by reference. Similar methods of preparations of biodegradable microencapsulated steroid hormones are used in U.S. Pat. No. 5,340,585 (36; Pike M and Spicer D V: Methods and formulations for use in treating benign gynecological disorders; U.S. Pat. No. 5,340,585; 1994) for the treatment of benign gynecological disorders and in U.S. Pat. No. 5,340,586 (34; Pike M and Spicer D V: Methods and formulations for use in treating oophorectomized women, U.S. Pat. No. 5,340,586; 1994) for use of treating oophorectomized women. They are also hereby incorporated by reference. Similarly, any of the many previously known prior art methods for the preparation of microencapsulated compositions could also be used for the preparation of microencapsulated steroid hormones and their synthetic derivatives as prostatic implants for the treatment and prevention of prostate cancer of this invention.

1. Preparation of Slow-Release Microcapsules Containing DES for Prostatic Implant

In accordance with one preferred embodiment for preparation of slow-release microcapsules containing DES for prostatic implant, the following method is adapted. 3 g of DES and 3 g of poly(dl-lactide-coglycolide) are dissolved in 18 g of methylene chloride and dispersed as stable emulsions of microdroplets in 58 g of wt % of aqueous poly(vinyl alcohol). Afterwards, 60% of the solvent methylene chloride was removed by evaporation. The DES containing microcapsules are removed by centrifugation. The sediment of microencapsulated DES is then resuspended in deionized water and filtered through a fine fritted-glass funnel by slow suction while continuously adding more deionized water to remove the residual methylene chloride. This filtered microencapsulated DES is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing including the follow up estimations of serum PSA levels after implanting a test dose as an indicator of response to such implant treatment, the extended period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity. Implants of such microcapsules filled with DES to the prostate by injection will maintain a steady rate of slow release of DES by diffusion and by biodegradation of the capsules. It will maintain the plasma concentration of DES as sufficient to exert its androgen suppressive, and androgen independent beneficial actions that are critical for the treatment of prostate cancer. Hence there is lesser systemic toxic effects of DES associated with such implants. Furthermore, this local higher concentration of DES will saturate the estrogen binding sites of prostate cancer.

2. Preparation of Slow-Release Microcapsules Containing Estradiol for Prostatic Implant

In accordance with one preferred embodiment for preparation of slow-release microcapsules containing estradiol for prostatic implant, the following method is adapted. 3 g of DES and 3 g of poly(dl-lactide-coglycolide) are dissolved in 18 g of methylene chloride and dispersed as stable emulsions of microdroplets in 58 g of wt % of aqueous poly(vinyl alcohol). Afterwards, 60% of the solvent methylene chloride is removed by evaporation. The estradiol containing microcapsules are removed by centrifugation. The sediment of microencapsulated estradiol is then resuspended in deionized water and filtered through a fine fritted-glass funnel by slow suction while continuously adding more deionized water to remove the residual methylene chloride. This filtered microencapsulated estradiol is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing and with the follow up estimations of serum PSA levels after implanting a test dose as an indicator of response to such implant treatment, the extended period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with estradiol to the prostate by injection maintain a steady rate of slow release of estradiol by diffusion and by biodegradation of the capsules. It maintains the plasma concentration of estradiol sufficient to exert its androgen suppressive, and androgen independent beneficial actions that are critical for the treatment of the prostate cancer. The concentration of estradiol in prostate is much higher than the systemic concentration but sufficient to suppress the hypothalamic pituitary axis mediated androgen synthesis. Hence there is lesser systemic toxic effects of estradiol associated with such implants. Furthermore, this local higher concentration of estradiol will saturate the estrogen binding sites of prostate cancer.

3. Preparation of Slow-Release Microcapsules Containing Iodinated Estradiol, Iodoestradiol for Prostatic Implant

In accordance with one preferred embodiment for preparation of slow-release microcapsules containing iodoestradiol for prostatic implant, the following method is adapted. 3 g of iodoestradiol and 3 g of poly(dl-lactide-coglycolide) are dissolved in 18 g of methylene chloride and dispersed as stable emulsions of microdroplets in 58 g of wt % of aqueous poly(vinyl alcohol). Afterwards, 60% of the solvent methylene chloride is removed by evaporation. The iodinated estradiol containing microcapsules are removed by centrifugation. The sediment of microencapsulated estradiol is then resuspended in deionized water and filtered through a fine flitted-glass funnel by slow suction while continuously adding more deionized water to remove the residual methylene chloride.

This filtered microencapsulated iodinated estradiol is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing and with the follow up estimations of serum PSA levels after implanting a test dose as an indicator of response to such implant treatment, the extended period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with iodoestradiol to the prostate by injection will maintain a steady rate of slow release of iodoestradiol by diffusion and by biodegradation of the capsules. It will maintain the plasma concentration of iodoestradiol sufficient to exert its androgen suppressive, and androgen independent beneficial actions that are critical for the treatment of prostate cancer. The concentration of iodoestradiol in prostate is much higher than it is in the systemic concentration but sufficient to suppress the hypothalamic pituitary axis mediated androgen synthesis. Hence there is lesser systemic toxic effects of iodoestradiol associated with such implants. Furthermore, this local higher concentration of iodoestradiol will saturate the estrogen binding sites of prostate cancer.

4. Preparation of Slow-Release Microcapsules Containing Progesterone for Prostatic Implant

In accordance with one preferred embodiment for preparation of slow-release microcapsules containing progesterone for prostatic implant, the following method is adapted. 3 g of progesterone and 3 g of poly(dl-lactide-coglycolide) are dissolved in 18 g of methylene chloride and dispersed as stable emulsions of microdroplets in 58 g of wt % of aqueous poly(vinyl alcohol). Afterwards, 60% of the solvent methylene chloride is removed by evaporation. The progesterone containing microcapsules are removed by centrifugation. The sediment of microencapsulated estradiol is then resuspended in deionized water and filtered through a fine fritted-glass funnel by slow suction while continuously adding more deionized water to remove the residual methylene chloride. This filtered microencapsulated progesterone is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing and with the follow up estimations of serum PSA levels after implanting a test dose as an indicator of response to such implant treatment, the extended period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with progesterone to the prostate by injection will maintain a steady rate of slow release of progesterone by diffusion and by biodegradation of the capsules. It will maintain the plasma concentration of progesterone as sufficient to exert its androgen suppressive, and androgen independent beneficial actions that are critical for the treatment of prostate cancer. The concentration of progesterone in the prostate is much higher than the systemic concentration but sufficient to suppress the hypothalamic pituitary axis mediated androgen synthesis. Hence there is lesser systemic toxic effects of progesterone associated with such implants.

5. Preparation of Slow-Release Microcapsules Containing Prednisolone for Prostatic Implant

In accordance with one preferred embodiment for preparation of slow-release microcapsules containing prednisolone for prostatic implant, the following method is adapted. 3 g of prednisolone and 3 g of poly(dl-lactide-coglycolide) are dissolved in 18 g of methylene chloride and dispersed as stable emulsions of microdroplets in 58 g of wt % of aqueous poly(vinyl alcohol). Afterwards, 60% of the solvent methylene chloride is removed by evaporation. The prednisolone containing microcapsules are removed by centrifugation. The sediment of microencapsulated estradiol is then resuspended in deionized water and filtered through a fine fritted-glass funnel by slow suction while continuously adding more deionized water to remove the residual methylene chloride. This filtered microencapsulated prednisolone is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing including the follow up estimations of serum PSA levels after implanting a test dose as an indicator of response to such implant treatment, the extended period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with prednisolone to the prostate by injection will maintain a steady rate of slow release of prednisolone by diffusion and by biodegradation of the capsules. It will maintain the plasma concentration of prednisolone sufficient to exert its androgen suppressive, and androgen independent beneficial actions that are critical for the treatment of prostate cancer. The concentration of prednisolone in prostate is much higher than the systemic concentration but sufficient to suppress the hypothalamic pituitary axis mediated androgen synthesis. Hence there is lesser systemic toxic effects of prednisolone associated with such implants.

6. Preparation of Slow-Release Microcapsules Containing Flutamide for Prostatic Implant

In accordance with one preferred embodiment for preparation of slow-release microcapsules containing flutamide for prostatic implant, the following method is adapted. 3 g of flutamide and 3 g of poly(dl-lactide-coglycolide) are dissolved in 18 g of methylene chloride and dispersed as stable emulsions of microdroplets in 58 g of wt % of aqueous poly(vinyl alcohol). Afterwards, 60% of the solvent methylene chloride is removed by evaporation. The flutamide containing microcapsules are removed by centrifugation. The sediment of microencapsulated estradiol is then resuspended in deionized water and filtered through a fine fritted-glass funnel by slow suction while continuously adding more deionized water to remove the residual methylene chloride. This filtered microencapsulated flutamide is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing including the follow up estimations of serum PSA levels after implanting a test dose as an indicator of response to such implant treatment, the longer period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with flutamide to the prostate by injection will maintain a steady rate of slow release of flutamide by diffusion and by biodegradation of the capsules. It will maintain the plasma concentration of flutamide sufficient to exert its androgen suppressive, and androgen independent beneficial actions that are critical for the treatment of prostate cancer. The concentration of flutamide in prostate is much higher than it is in the systemic concentration but sufficient to suppress the hypothalamic pituitary axis mediated androgen synthesis. Hence there is lesser systemic toxic effects of flutamide associated with such implants. Furthermore, this local higher concentration of flutamide will saturate the androgen binding sites of prostate cancer.

7. Preparation of Slow-Release Microcapsules Containing Estramustine for Prostatic Implants

In accordance with one preferred embodiment for preparation of slow-release microcapsules containing estramustine for prostatic implant, the following method is adapted. 3 g of estramustine and 3 g of poly(dl-lactide-coglycolide) are dissolved in 18 g of methylene chloride and dispersed as stable emulsions of microdroplets in 58 g of wt % of aqueous poly(vinyl alcohol). Afterwards, 60% of the solvent methylene chloride is removed by evaporation. The estramustine containing microcapsules are removed by centrifugation. The sediment of microencapsulated estradiol is then resuspended in deionized water and filtered through a fine fritted-glass funnel by slow suction while continuously adding more deionized water to remove the residual methylene chloride. This filtered microencapsulated estramustine is then sieved through a stainless-steel screen. The microcapsules comprising 50 wt % is then suspended in sterile normal saline.

For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing including the follow up estimations of serum PSA levels after implanting a test dose as an indicator of response to such implant treatment, the longer period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with estramustine to the prostate by injection will maintain a steady rate of slow release of estramustine by diffusion and by biodegradation of the capsules. It will maintain the plasma concentration of estramustine and its metabolic products sufficient to exert its androgen suppressive, and androgen independent beneficial actions.

The concentration o f estramustine diffused from the capsule is much higher in the prostate than its systemic concentration. Hence there is lesser systemic toxic effects of estramustine associated with such implants. Estramustine preferentially binds to the estramustine binding protein that is abundantly present in the prostatic epithelial cells. Its estrogenic activity and microtubular inhibitory properties mediate its cytotoxicity. The local high concentration of estramustine diffused from the implanted capsules will saturate the estramustine binding sites of the prostate cancer. Hence it is a much more efficient treatment of prostate cancer than when estramustine is administered orally. The usually recommended dose of estramustine for the treatment of prostate cancer is 10-16 mg per kg body weight per day. Therefore, the dose for a patient weighing 70 kg would be about 1000 mg. Because of this high dose of orally administered estramustine, it has higher systemic toxicity. The concentration reaching the prostate by oral administration is much lower than by its implant to the prostate. Therefore, there will not be sufficient estramustine to bind all of the estramustine binding protein of the prostate and the prostate cancer. Hence in spite of the high dose of the orally administered estramustine it is less effective to inhibit tumor growth. This may be the reason why the estramustine is not as effective as it was thought to be to control prostate cancer.

The major metabolites of estramustine are the estrone and estradiol analogues. Like the estrogen derivative of estramustine, the estrone derivative of estramustine also binds to its binding proteins in the prostatic epithelial cells. The high affinity prostatic epithelial cell bound estramustine is metabolized to estrogen. Thus there will be abundance of such metabolized estrogen within the cells, it will saturate the cell's capacity to bind estrogen.

It thus enhances the beneficial actions of estramustine on prostate cancer but with lesser toxicity as compared to systemically administered estrogen.

8. Preparation of Slow-Release Microcapsules Containing DES and Prednisolone for Prostatic Implant.

In accordance with one preferred embodiment for preparation of slow-release microcapsules containing DES and prednisolone for prostatic implant, the following method is adapted. 3 g of DES, 3 g of prednisolone and 6 g of poly(dl-lactide-coglycolide) are dissolved in 36 g of methylene chloride and dispersed as stable emulsions of microdroplets in 116 g of wt % of aqueous poly(vinyl alcohol). Afterwards, 60% of the solvent methylene chloride is removed by evaporation. The DES and prednisolone estradiol containing microcapsules are removed by centrifugation. The sediment of microencapsulated estradiol is then resuspended in deionized water and filtered through a fine fritted-glass funnel by slow suction while continuously adding more deionized water to remove the residual methylene chloride. This filtered microencapsulated DES and prednisolone is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing including the follow up estimations of serum PSA levels after implanting a test dose as an indicator of response to such implant treatment, the extended period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with DES and prednisolone to the prostate by injection will maintain a steady rate of slow release of DES and prednisolone by diffusion and by biodegradation of the capsules. It will maintain the plasma concentration of DES and prednisolone sufficient to exert their androgen suppressive, and androgen independent beneficial actions that are helpful for the treatment of prostate cancer. The concentrations of DES and prednisone diffused from the capsules are much higher in the prostate than their systemic concentrations. Hence there is lesser systemic toxic effects of DES and prednisolone associated with such implants. Prednisolone also suppresses the adrenal synthesis of androgens including adrenal testosterone synthesis. Furthermore, this local higher concentration of DES will saturate the estrogen binding sites of prostate cancer. This combination implants of DES and prednisolone enhances tumor control by their combined estrogenic and androgen suppressive actions. Such a combination is effective in both hormone dependent and hormone refractory prostate cancer.

9. Preparation of Slow-Release Microcapsules Containing DES and Flutamide for Prostatic Implant.

In accordance with one preferred embodiment for preparation of slow-release microcapsules containing DES and flutamide for prostatic implant, the following method is adapted. 3 g of DES, 3 g of flutamide and 6 g of poly(dl-lactide-coglycolide) are dissolved in 36 g of methylene chloride and dispersed as stable emulsions of microdroplets in 116 g of wt % of aqueous poly(vinyl alcohol). Afterwards, 60% of the solvent methylene chloride is removed by evaporation. The DES and flutamide containing microcapsules are removed by centrifugation. The sediment of microencapsulated estradiol is then resuspended in deionized water and filtered through a fine fritted-glass funnel by slow suction while continuously adding more deionized water to remove the residual methylene chloride. This filtered microencapsulated DES and flutamide is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing including the follow up estimations of serum PSA levels after implanting a test dose as an indicator of response to such implant treatment, the extended period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with DES and flutamide to the prostate by injection will maintain a steady rate of slow release of DES and prednisolone by diffusion and by biodegradation of the capsules. It will maintain the plasma concentration of DES and flutamide sufficient to exert their androgen suppressive, and androgen independent beneficial actions that are helpful for the treatment of prostate cancer. The concentrations of DES and flutamide diffused from the capsules are much higher in the prostate than their systemic concentration. Hence there is lesser systemic toxic effects of DES and flutamide associated with such implants. Furthermore, this local higher concentration of DES and flutamide will saturate both the estrogen and androgen binding sites of prostate cancer. This combination implants of DES and flutamide enhances tumor control by their combined estrogenic and androgen suppressive actions. DES is also known to be effective in hormone refractory prostate cancer.

10 Preparation of Slow-Release Microcapsules Containing DES and Progesterone for Prostatic Implant.

In accordance with one preferred embodiment for preparation of slow-release microcapsules containing DES and progesterone for prostatic implant, the following method is adapted. 3 g of DES, 3 g of progesterone and 6 g of poly(dl-lactide-coglycolide) are dissolved in 36 g of methylene chloride and dispersed as stable emulsions of microdroplets in 116 g of wt % of aqueous poly(vinyl alcohol). Afterwards, 60% of the solvent methylene chloride is removed by evaporation. The DES and progesterone containing microcapsules are removed by centrifugation. The sediment of microencapsulated estradiol is then resuspended in deionized water and filtered through a fine fritted-glass funnel by slow suction while continuously adding more deionized water to remove the residual methylene chloride. This filtered microencapsulated DES and progesterone is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing including the follow up estimations of serum PSA levels after implanting a test dose as an indicator of response to such implant treatment, the extended period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with DES and progesterone to the prostate by injection will maintain a steady rate of slow release of DES and progesterone prednisolone by diffusion and by biodegradation of the capsules. It will maintain the plasma concentration of DES and progesterone sufficient to exert their androgen suppressive, and androgen independent beneficial actions that are helpful for the treatment of prostate cancer. The concentrations of DES and progesterone diffused from the capsules are much higher in the prostate than their systemic concentration. Hence there is lesser systemic toxic effects of DES and progesterone associated with such implants. Furthermore, this local higher concentration of DES will saturate the estrogen binding sites of prostate cancer. This combination implants of DES and progesterone enhances tumor control by their combined estrogenic and androgen suppressive actions. Such a combination is an effective treatment for prostate cancer that are both hormone dependent and hormone refractory but still with residual androgen sensitivity and or sensitivity to other hormones like progesterone. DES is also known to be effective in hormone refractory prostate cancer.

11 Preparation of Slow-Release Microcapsules Containing Estradiol and Prednisolone for Prostatic Implant.

In accordance with one preferred embodiment for preparation of slow-release microcapsules containing estradiol and prednisolone for prostatic implant, the following method is adapted. 3 g of estradiol, 3 g of prednisolone and 6 g of poly(dl-lactide-coglycolide) are dissolved in 36 g of methylene chloride and dispersed as stable emulsions of microdroplets in 116 g of wt % of aqueous poly(vinyl alcohol). Afterwards, 60% of the solvent methylene chloride is removed by evaporation. The estradiol and prednisolone containing microcapsules are removed by centrifugation. The sediment of microencapsulated estradiol is then resuspended in deionized water and filtered through a fine fritted-glass funnel by slow suction while continuously adding more deionized water to remove the residual methylene chloride. This filtered microencapsulated estradiol and prednisolone is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing including the follow up estimations of serum PSA levels after implanting a test dose as an indicator of response to such implant treatment, the extended period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with estradiol and prednisolone to the prostate by injection will maintain a steady rate of slow release of estradiol and prednisolone by diffusion and by biodegradation of the capsules. It will maintain the plasma concentration of estradiol and prednisolone sufficient to exert their androgen suppressive, and androgen independent beneficial actions that are helpful for the treatment of prostate cancer. The concentrations of estradiol and prednisone diffused from the capsules are much higher in the prostate than their systemic concentration. Hence there is lesser systemic toxic effects of estradiol and prednisolone associated with such implants. Prednisolone also suppresses the adrenal synthesis of androgens including adrenal testosterone synthesis. Furthermore, this local higher concentration of estradiol will saturate the estrogen binding sites of prostate cancer. This combination implants of estradiol and prednisolone enhances tumor control by their combined estrogenic and androgen suppressive actions. Such a combination is an effective treatment for prostate cancer that are both hormone dependent and hormone refractory but still with residual androgen sensitivity and or sensitivity to other hormones like prednisolone.

12 Preparation of Slow-Release Microcapsules Containing Estradiol and Progesterone for Prostatic Implant.

In accordance with one preferred embodiment for preparation of slow-release microcapsules containing estradiol and progesterone for prostatic implant, the following method is adapted. 3 g of estradiol, 3 g of progesterone and 6 g of poly(dl-lactide-coglycolide) are dissolved in 36 g of methylene chloride and dispersed as stable emulsions of microdroplets in 116 g of wt % of aqueous poly(vinyl alcohol). Afterwards, 60% of the solvent methylene chloride is removed by evaporation. The estradiol and progesterone containing microcapsules are removed by centrifugation. The sediment of microencapsulated estradiol is then resuspended in deionized water and filtered through a fine fritted-glass funnel by slow suction while continuously adding more deionized water to remove the residual methylene chloride. This filtered microencapsulated estradiol and progesterone is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing including the follow up estimations of serum PSA levels after implanting a test dose as an indicator of response to such implant treatment, the extended period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with estradiol and progesterone to the prostate by injection will maintain a steady rate of slow release of estradiol and progesterone by diffusion and by biodegradation of the capsules. It will maintain the plasma concentrations of estradiol and progesterone sufficient to exert their androgen suppressive, and androgen independent beneficial actions that are helpful for the treatment of prostate cancer. The concentrations of estradiol and progesterone diffused from the capsules are much higher in the prostate than their systemic concentration. Hence there is lesser systemic toxic effects of estradiol and progesterone associated with such implants. Furthermore, this local higher concentration of estradiol will saturate the estrogen binding sites of prostate cancer. This combination implants of estradiol and progesterone enhances tumor control by their combined estrogenic and androgen suppressive actions. Such a combination is an effective treatment for prostate cancer that are both hormone dependent and hormone refractory but with still with residual androgen sensitivity and or sensitivity to other hormones like progesterone.

13 Preparation of Slow-Release Microcapsules Containing Estradiol and Flutamide for Prostatic Implant.

In accordance with one preferred embodiment for preparation of slow-release microcapsules containing estradiol and flutamide for prostatic implant, the following method is adapted. 3 g of estradiol, 3 g of flutamide and 6 g of ply(dl-lactide-coglycolide) are dissolved in 36 g of methylene chloride and dispersed as stable emulsions of microdroplets in 116 g of wt % of aqueous poly(vinyl alcohol). Afterwards, 60% of the solvent methylene chloride is removed by evaporation. The estradiol and flutamide containing microcapsules are removed by centrifugation. The sediment of microencapsulated estradiol is then resuspended in deionized water and filtered through a fine fritted-glass funnel by slow suction while continuously adding more deionized water to remove the residual methylene chloride. This filtered microencapsulated estradiol and flutamide is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing including the follow up estimations of serum PSA levels after implanting a test dose as an indicator of response to such implant treatment, the extended period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with estradiol and flutamide to the prostate by injection will maintain a steady rate of slow release of estradiol and flutamide by diffusion and by biodegradation of the capsules. It will maintain the plasma concentrations of estradiol and flutamide sufficient to exert their androgen suppressive, and androgen independent beneficial actions that are helpful for the treatment of prostate cancer.

The concentration of estradiol and flutamide diffused from the capsule is much higher in the prostate than their systemic concentrations. Hence there is lesser systemic toxic effects of estradiol and flutamide associated with such implants. The local higher concentration of estradiol will saturate the estrogen binding sites of prostate cancer. Furthermore, since flutamide binds to androgen receptor sites competitively with testosterone, this local higher concentration of flutamide will saturate the testosterone binding sites of prostate cancer. Hence such a combination of estradiol and flutamide is a much more efficient treatment of prostate cancer than when flutamide is administered orally. Because of these high dose of orally administered estradiol and flutamide, they have much more systemic toxicity than by this implant treatment. Their concentrations reaching the prostate by the oral administration is much lower than those achieved by their prostatic implants. There will not be sufficient estrogen and flutamide to bind all of the estrogen and testosterone receptor sites of the prostate and the prostate cancer. Hence orally administered estradiol and flutamide are less effective to inhibit the androgen dependent growth of prostate cancer. At present flutamide is administered orally. This combination implants of estradiol and flutamide enhances tumor control by their combined estrogenic and androgen suppressive actions. Such a combination is an effective treatment for prostate cancer.

14 Preparation of Slow-Release Microcapsules Containing Estramustine and Prednisolone for Prostatic Implant

In accordance with one preferred embodiment for preparation of slow-release microcapsules containing estramustine and prednisone for prostatic implant, the following method is adapted. 3 g of estramustine, 3 g of prednisolone and 6 g of poly(dl-lactide-coglycolide) are dissolved in 36 g of methylene chloride and dispersed as stable emulsions of microdroplets in 116 g of wt % of aqueous poly(vinyl alcohol). Afterwards, 60% of the solvent methylene chloride is removed by evaporation. The estramustine and prednisolone containing microcapsules are removed by centrifugation. The sediment of microencapsulated estradiol is then resuspended in deionized water and filtered through a fine fritted-glass funnel by slow suction while continuously adding more deionized water to remove the residual methylene chloride. This filtered microencapsulated estramustine and prednisolone is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing including the follow up estimations of serum PSA levels after implanting a test dose as an indicator of response to such implant treatment, the extended period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with estramustine and prednisolone to the prostate by injection will maintain a steady rate of slow release of estradiol and prednisolone by diffusion and by biodegradation of the capsules. It will maintain the plasma concentrations of estradiol and prednisolone sufficient to exert their androgen suppressive, and androgen independent beneficial actions that are helpful for the treatment of prostate cancer.

The concentrations of estramustine and prednisolone diffused from the capsule are much higher in the prostate than their systemic concentrations. Hence there is lesser systemic toxic effects of estramustine and prednisolone associated with such implants. Estramustine preferentially binds to the estramustine binding protein that is abundantly present in the prostatic epithelial cells. Its estrogenic activity and its microtubular inhibitory properties mediate its cytotoxicity. The local high concentration of estramustine diffused from the implanted capsules will saturate the estramustine binding sites of the prostate cancer with high affinity. Hence it is a much more efficient treatment of prostate cancer than when estramustine is administered orally. The usual recommended dose of estramustine for the treatment of prostate cancer is 10-16 mg per kg body weight per day. Therefore, the dose for a patient weighing 70 kg would be about 1000 mg. Because of this high dose of orally administered estramustine, it has much systemic toxicity. Its concentration reaching the prostate by the oral administration is much lower than those achieved by its implant to the prostate. Therefore, there is not sufficient estramustine to bind all of the estramustine binding protein of the prostate and the prostate cancer. Hence in spite of the high dose of the orally administered estramustine it is less effective to inhibit tumor growth. This may be the reason why the estramustine is not as effective as it was thought to be to control prostate cancer.

The major metabolites of estramustine are the estrone and estramustine analogues. Like the estrogen derivative of estramustine, the estrone derivative of estramustine will bind to its binding proteins in the prostatic epithelial cells. Because implants are within the prostate, high concentration of estramustine will be diffused from the implanted capsules to the prostate. This facilitates a much higher concentration of estramustine in the prostate than that would reach the prostate after its oral administration. Therefore, its cytotoxic actions on prostate cancer are much greater than when it is administered orally. Since the high affinity prostatic epithelial cell bound estramustine is metabolized to estrogen and since there will be abundant such metabolized estrogen within the cells, it will saturate the cell's capacity to bind estrogen to its estrogen binding sites. It will thus enhance the beneficial actions of estrogen on prostate cancer but with lesser toxicity as compared to systemically administered estrogen. Prednisolone suppresses the adrenal synthesis of androgens including the adrenal testosterone. It is also very effective in hormone refractory prostate cancer. This combination implants of estramustine and prednisolone enhances tumor control by their combined cytotoxicity and androgen suppressive actions. Such a combination is effective in both hormone dependent and hormone refractory prostate cancer.

15 Preparation of Slow-Release Microcapsules Containing Estramustine and Flutamide for Prostatic Implant

In accordance with one preferred embodiment for preparation of slow-release microcapsules containing estramustine and flutamide for prostatic implant, the following method is adapted. 3 g of estramustine, 3 g of flutamide and 6 g of poly(dl-lactide-coglycolide) are dissolved in 36 g of methylene chloride and dispersed as stable emulsions of microdroplets in 116 g of wt % of aqueous poly(vinyl alcohol). Afterwards, 60% of the solvent methylene chloride is removed by evaporation. The estramustine and flutamide containing microcapsules are removed by centrifugation. The sediment of microencapsulated estradiol is then resuspended in deionized water and filtered through a fine fritted-glass funnel by slow suction while continuously adding more deionized water to remove the residual methylene chloride. This filtered microencapsulated estramustine and flutamide is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing including the follow up estimations of serum PSA levels after implanting a test dose as an indicator of response to such implant treatment, the extended period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with estramustine and flutamide to the prostate by injection will maintain a steady rate of slow release of estradiol and flutamide by diffusion and by biodegradation of the capsules. It will maintain the plasma concentrations of estradiol and flutamide sufficient to exert their androgen suppressive, and androgen independent beneficial actions that are helpful for the treatment of prostate cancer. The concentrations of estramustine and flutamide diffused from the capsule are much higher in the prostate than their systemic concentrations. Hence there is lesser systemic toxicity from implanting capsules containing estramustine and flutamide to the prostate. This combination of estramustine and flutamide enhances tumor control by their combined cytotoxicity and androgen suppressive actions. Such combination is effective in both hormone dependent and hormone refractory prostate cancer.

16 Preparation of Slow-Release Microcapsules Containing Estramustine and Progesterone for Prostatic Implant

In accordance with one preferred embodiment for preparation of slow-release microcapsules containing estramustine and progesterone for prostatic implant, the following method is adapted. 3 g of estramustine, 3 g of progesterone and 6 g of poly(dl-lactide-coglycolide) are dissolved in 36 g of methylene chloride and dispersed as stable emulsions of microdroplets in 116 g of wt % of aqueous poly(vinyl alcohol). Afterwards, 60% of the solvent methylene chloride is removed by evaporation. The estramustine and progesterone containing microcapsules are removed by centrifugation. The sediment of microencapsulated estradiol is then resuspended in deionized water and filtered through a fine fritted-glass funnel by slow suction while continuously adding more deionized water to remove the residual methylene chloride. This filtered microencapsulated estramustine and progesterone is then sieved through a stainless-steel screen.

The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing including the follow up estimations of serum PSA levels after implanting a test dose as an indicator of response to such implant treatment, the extended period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with estramustine and progesterone to the prostate by injection will maintain a steady rate of slow release of estradiol and progesterone by diffusion and by biodegradation of the capsules. It will maintain the plasma concentrations of estradiol and progesterone sufficient to exert their androgen suppressive, and androgen independent beneficial actions that are helpful for the treatment of prostate cancer. The concentrations of estramustine and progesterone diffused from the capsule are much higher in the prostate than their systemic concentrations. Hence there is lesser systemic toxicity from implanting capsules containing estramustine and progesterone to the prostate. This combination of estramustine and progesterone enhances the tumor control by their combined cytotoxicity and androgen suppressive actions. Such combination is effective in both hormone dependent and hormone refractory prostate cancer.

17 Preparation of Slow-Release Microcapsules Containing DES, Prednisolone and Flutamide for Prostatic Implant

In accordance with one preferred embodiment for preparation of slow-release microcapsules containing DES, prednisolone and flutamide for prostatic implant, the following method is adapted. 3 g of DES, 3 g of prednisolone, 3 g of flutamide and 9 g of poly(dl-lactide-coglycolide) are dissolved in 54 g of methylene chloride and dispersed as stable emulsions of microdroplets in 174 g of wt % of aqueous poly(vinyl alcohol). Afterwards, 60% of the solvent methylene chloride is removed by evaporation. The DES, prednisolone and flutamide containing microcapsules are removed by centrifugation. The sediment of microencapsulated estradiol is then resuspended in deionized water and filtered through a fine fritted-glass funnel by slow suction while continuously adding more deionized water to remove the residual methylene chloride. This filtered microencapsulated DES, prednisolone and flutamide is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing including the follow up estimations of serum PSA levels after implanting a test dose as an indicator of response to such implant treatment, the extended period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with DES, prednisolone and flutamide to the prostate by injection will maintain a steady rate of slow release of DES, prednisolone and flutamide by diffusion and by biodegradation of the capsules. It will maintain the plasma concentrations of DES, prednisolone and flutamide sufficient to exert their androgen suppressive, and androgen independent beneficial actions that are helpful for the treatment of prostate cancer. The concentrations of DES, prednisolone and flutamide diffused from the capsule are much higher in the prostate than their systemic concentrations. Hence there is lesser systemic toxicity from implanting capsules containing DES, prednisolone and flutamide to the prostate. This triple drug combinations of DES, prednisolone and flutamide enhances prostatic tumor control by their combined cytotoxicity and androgen suppressive actions. Such combination is effective in both hormone dependent and hormone refractory prostate cancer.

18 Preparation of Slow-Release Microcapsules Containing DES, Progesterone and Flutamide for Prostatic Implant

In accordance with one preferred embodiment for preparation of slow-release microcapsules containing DES, progesterone and flutamide for prostatic implant, the following method is adapted. 3 g of DES, 3 g of progesterone, 3 g of flutamide and 9 g of poly(dl-lactide-coglycolide) are dissolved in 54 g of methylene chloride and dispersed as stable emulsions of microdroplets in 174 g of wt % of aqueous poly(vinyl alcohol). Afterwards, 60% of the solvent methylene chloride is removed by evaporation. The DES, progesterone and flutamide containing microcapsules are removed by centrifugation. The sediment of microencapsulated estradiol is then resuspended in deionized water and filtered through a fine fritted-glass funnel by slow suction while continuously adding more deionized water to remove the residual methylene chloride. This filtered microencapsulated DES, progesterone and flutamide is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing including the follow up estimations of serum PSA levels after implanting a test dose as an indicator of response to such implant treatment, the extended period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with DES, progesterone and flutamide to the prostate by injection will maintain a steady rate of slow release of DES, progesterone and flutamide by diffusion and by biodegradation of the capsules. It will maintain the plasma concentrations of DES, progesterone and flutamide sufficient to exert their androgen suppressive, and androgen independent beneficial actions that are helpful for the treatment of prostate cancer. This triple drug combination of DES, progesterone and flutamide enhances the prostatic tumor control by their combined cytotoxicity and androgen suppressive actions. Such combination is effective in both hormone dependent and hormone refractory prostate cancer.

19 Preparation of Slow-Release Microcapsules Containing Estradiol, Prednisolone and Flutamide for Prostatic Implant

In accordance with one preferred embodiment for preparation of slow-release microcapsules containing estradiol, prednisolone and flutamide for prostatic implant, the following method is adapted. 3 g of estradiol, 3 g of prednisolone, 3 g of flutamide and 9 g of poly(dl-lactide-coglycolide) are dissolved in 54 g of methylene chloride and dispersed as stable emulsions of microdroplets in 174 g of wt % of aqueous poly(vinyl alcohol). Afterwards, 60% of the solvent methylene chloride is removed by evaporation. The estradiol, prednisone and flutamide containing microcapsules are removed by centrifugation. The sediment of microencapsulated estradiol is then resuspended in deionized water and filtered through a fine fritted-glass funnel by slow suction while continuously adding more deionized water to remove the residual methylene chloride. This filtered microencapsulated estradiol, prednisone and flutamide is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing including the follow up estimations of serum PSA levels after implanting a test dose as an indicator of response to such implant treatment, the extended period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with estradiol, prednisolone and flutamide to the prostate by injection will maintain a steady rate of slow release of estradiol, prednisolone and flutamide by diffusion and by biodegradation of the capsules. It will maintain the plasma concentrations of estradiol, prednisolone and flutamide sufficient to exert their androgen suppressive, and androgen independent beneficial actions that are helpful for the treatment of prostate cancer. The concentrations of estradiol, prednisolone and flutamide diffused from the capsule are much higher in the prostate than their systemic concentration. Hence there is lesser systemic toxicity from implanting capsules containing estradiol, prednisolone and flutamide to the prostate. This triple drug combination of estradiol, prednisolone and flutamide enhances prostatic tumor control by their combined cytotoxicity and androgen suppressive actions. Such combination is effective in both hormone dependent and hormone refractory prostate cancer.

20 Preparation of Slow-Release Microcapsules Containing Estradiol, Progesterone and Flutamide for Prostatic Implant

In accordance with one preferred embodiment for preparation of slow-release microcapsules containing estradiol, progesterone and flutamide for prostatic implant, the following method is adapted. 3 g of estradiol, 3 g of progesterone, 3 g of flutamide and 9 g of poly(dl-lactide-coglycolide) are dissolved in 54 g of methylene chloride and dispersed as stable emulsions of microdroplets in 174 g of wt % of aqueous poly(vinyl alcohol). Afterwards, 60% of the solvent methylene chloride is removed by evaporation. The estradiol, progesterone and flutamide containing microcapsules are removed by centrifugation. The sediment of microencapsulated estradiol is then resuspended in deionized water and filtered through a fine fritted-glass funnel by slow suction while continuously adding more deionized water to remove the residual methylene chloride. This filtered microencapsulated estradiol, progesterone and flutamide is then sieved through a stainless-steel screen. The microcapsules comprising 50 wt % is then suspended in sterile normal saline. For making locally chelating implants when it comes in contact with tissue, the microcapsules are suspended in a mixture of sterile normal saline, a local anesthetic and ethanol. The microcapsule preparations are sterilized by any of the known convenient method of sterilization. It is then dispensed into sterile syringes under sterile conditions for injections.

Based upon the need of a particular patient and on clinical testing including the follow up estimations of serum PSA levels after implanting a test dose as an indicator of response to such implant treatment, the extended period implant dose is adjusted to achieve the desired daily therapeutic concentration. Because these implants are within the prostate, the concentration of its contents diffused from the capsules is much higher in the prostate than its systemic concentration and hence it exerts its maximum therapeutic effects in the prostate with no significant systemic toxicity.

Implants of such microcapsules filled with estradiol, progesterone and flutamide to the prostate by injection will maintain a steady rate of slow release of estradiol, progesterone and flutamide by diffusion and by biodegradation of the capsules. It will maintain the plasma concentrations of estradiol, progesterone and flutamide sufficient to exert their androgen suppressive, and androgen independent beneficial actions that are helpful for the treatment of prostate cancer. The concentrations of estradiol, progesterone and flutamide diffused from the capsule are much higher in the prostate than their systemic concentration. Hence there is lesser systemic toxicity from implanting capsules containing estradiol, progesterone and flutamide to the prostate. This triple drug combination of estradiol, progesterone and flutamide enhances prostatic tumor control by their combined cytotoxicity and androgen suppressive actions. Such combination is effective in both hormone dependent and hormone refractory prostate cancer.

### Preferred Embodiment—Operation

Pre- and Post-Hormone Implant PSA Levels as a Guide for Follow Up and Further Treatment for Selected Patients with Early Stage Prostate Cancer

PSA is a glucoprotein that is produced only by the prostatic epithelium. Serum PSA is elevated in prostate cancer. PSA level is extremely useful to assess the tumor response to treatment. The age specific normal reference values for PSA is 2.5 ng/ml at age 40 to 49, 3.5 ng/ml at age 50 to 59, 4.5 ng/ml at ages 60 to 69 and 5.5 ng/ml at ages 70-79. The level of pretreatment serum PSA of patients with prostate cancer is an important prognostic indicator. Even those patients with the apparent normal PSA level, a dynamic increase of PSA would be an indication of developing prostate cancer. Prognostically, patients with PSA level of greater than 20 ng/ml is considered as high-risk patients. Their prognosis is similar to those with locally advanced prostate cancer.

After complete removal of the prostate by radical prostatectomy, no measurable PSA will be detected. Three weeks after surgery for early stage prostate cancer, the presence of residual PSA is indicative of incomplete removal of the prostate. After radiation therapy, measurable amount of serum PSA will be found. It is because of the still present prostate. A rising PSA level after radiation therapy is indicative of biochemical relapse of the prostate cancer. Hormonal treatment of prostate cancer also controls the serum PSA level. Like radiation, the hormonal treatment of prostate cancer reduces the serum PSA level. With the hormonal treatment, the PSA can be brought to a nadir value of less than 1 ng/ml. This includes patients with far-advanced prostate cancer and associated pre-treatment PSA level of over 300 ng/ml.

As in radiation therapy, the pre and post hormone implant PSA levels indicate biochemical tumor control as a result of hormone induced tumor suppression. PSA is an easily available laboratory test. Like in radiation therapy, a rise in serum PSA after androgen suppressive hormone implants to the prostate would indicate tumor growth and biochemical failure. Early stage prostate cancer treated by conventional low dose radiation combined with androgen suppressive treatment renders lower rate of tumor-positive biopsies. This lower rate of positive tumor biopsies is comparable with the treatment higher dose radiation alone. The addition of hormone with radiation facilitates same rate of negative tumor biopsies as with higher dose radiation treatment alone.

Two years after hormone implants alone to early stage prostate cancer, there would also be a decrease in tumor positive biopsies. After hormone implant, if a patient maintains a stable normal PSA and negative or most favorable histology, then such a patient may need only continued careful follow up. If a patient is found to have low or intermediate grade early stage prostate cancer and increasing PSA levels two years after the hormonal implant, then such a patient can still be treated by surgery or radiation therapy without any adverse clinical outcome. If a patient remains clinically controlled with normal PSA, follow up biopsies would determine the presence or absence of residual tumor and or any changes in the tumor characteristics including its Gleason grade. If there are adverse changes in the tumor status by biopsy or increasing PSA, then treatments with surgery or radiation therapy can be followed

Prophylactic Radiation to Breast before Prostatic Hormonal Implant to Prevent Gynecomastia

Under the estrogenic hormonal influence, the male breast will become tender and painful with accompanying enlargement of the breast (gynecomastia). If prophylactic radiation to breast is given two weeks before the estrogenic hormonal treatment, this gynecomastia can be prevented. Generally, before the hormonal treatment, 3-5 Gy external beam radiation with 9 MeV electrons or cobalt-60 to 4MV photon beam daily for three treatments is given to both breasts to prevent the development of gynecomastia. Similar prophylactic radiation to both breasts is given two weeks before the hormonal implant to the prostate to prevent gynecomastia.

Test Dose Implant of Androgen Suppressive Formulations

A test dose of androgen suppressive formulations encapsulated in Silastic capsules is implanted to the prostate or subcutaneousely with the aid of a trocar and an obturator. These Silastic capsules are made not biodegradable ones. For subcutaneous implant, after making a small incision of the skin of the inner surface of the upper arm under aseptic and local anesthetic conditions, the trocar with the obturator is inserted subcutaneousely to a distance of about 4-cms from the incision. The obturator is then withdrawn and the capsule is inserted into the trocar and it is advanced towards the tip of the trocar with the obturator and then the trocar is withdrawn just enough to lodge the implant subcutaneousely. If multiple capsules are to be implanted, they are placed in a fanlike manner using the same skin incision. Similarly, a test dose implant is implanted directly to the prostate as described for the prostatic implants. When prostatic implants are made, a 2 mm sized metallic marker is also inserted to the trocar and both the capsule and the marker are implanted to the same site. This metallic marker helps to identify the implant site in the prostate by diagnostic imaging. Four weeks after the implant, serum PSA level is determined at monthly intervals for about three to six months to assess the biochemical tumor control. It should have reached to a nadir value of about 1 ng per ml or lower. The serum level of the androgen suppressive formulation and the testosterone are also determined to make approximate dose estimation for the permanent implant in the prostate.

If any major adverse effect associated the test dose implant is observed, the subcutaneous test dose implant is removed by making an incision to the skin at the implant site under aseptic and local anesthetic conditions and gently palpating and withdrawing the implant with a forceps. Prostatic implants are removed by limited surgical approach. If there are any major adverse effects associated with such hormonal implant formulation, the permanent implant is not elected.

Methods of Implanting the Hormonal Compositions to the Prostate

There are a number of methods used to make implants to the prostate. They are mostly described for the interstitial radioactive seed implants, generally known as the brachytherapy for prostate cancer. The Greek “brachy” means regional. Hormone implants to prostate is also a regional therapy hence, it is the hormonal brachytherapy for prostate cancer. Because of the radioactivity in the brachytherapy with radioactive seeds and the need to derive accurate dose computations it is a complex procedure. Hormonal implantation to the prostate however is much simpler procedure and it does not need the complex and elaborate methods as for brachytherapy with radioactive seeds. However the same general approach as for the interstitial radioactive seed implant is adapted for the hormonal implants to the prostate. These includes the well known methods of retropubic implants, trans perennial implant, transrectal ultrasound based visualization of the prostate and implantation, computed tomography based visualization of the prostate and implantation or by surgically exposing and free hand implanting. Similar to the Silastic capsule implant methods described for the subcutaneous test dose implant above with a trocar and an obturator, the hormonal formulation encapsulated in Silastic capsules or the hormone fused with a lipoid carrier and with a metallic marker is placed in the prostate. The microcapsule implants are injected to the prostate with a syringe and needle.

Concomitant Hormonal Implant Treatment With Radiation Therapy

The concomitant hormonal treatment with radiation is known to improve the treatment outcome of prostate cancer. Addition of androgen suppressive hormonal treatment combined with 64.8 Gy external radiation is equivalent to the higher 81 Gy dose external beam radiation without the added hormonal treatment. Pre-interstitial radioactive seed implant treatment of a prostate with cancer and with a prostatic volume greater than 50 cc, the androgen suppressive treatment with LHRH is generally used. Such LHRH treatment will reduce about 40 per cent of the initial prostatic volume. Such prostatic volume reduction will facilitate better placement of radioactive seeds within the prostate, a necessary requirement for brachytherapy. The slow constant rate hormonal release from the hormonal implants to the prostate combined with radiation is also is an effective means to control the prostate cancer and its cure. Furthermore, this facilitates cure and control of prostate cancer with lesser and better tolerated dose of radiation.

The hormonal implants to the prostate could be done either before or concomitantly with the interstitial radioactive seed implants to the prostate. An added advantage of such combined hormonal implant and external radiation therapy is that it also effectively controls regional lymph node metastasis since these hormonal compositions from the biodegrading implants will be carried to the regional lymph nodes by the macrophages. In the case of interstitial radiotherapy, such added advantage of radiation at the site of regional lymph nodes is not possible. The very weak low energy radiation from the radioactive seeds of interstitial radiation therapy is confined within the prostate and will not reach the distant regional lymph nodes of the prostate.

Prophylactic Hormonal Treatment of Prostate Cancer

Since prostate cancer is androgen dependent, androgen suppressive measures lends itself as a prophylactic measure to arrest development of prostate cancer. Very low doses of an androgen suppressive hormone like an estrogenic substance would suppress the development and or further growth and differentiation of a clone of cells otherwise destined to become the small early focus of a developing prostate cancer. Prostate cancer is a disease of elderly men with an average age of 72 years at diagnosis. Small dose slow-release androgen suppressive hormonal implants to the prostate that will maintain the serum prostate specific antigen to a nadir value of 1 ng per ml or lower and without systemic toxicity is an effective hormonal prophylactic treatment.

Conclusions, Ramifications, and Scope

Although the description above contains much specificity, these should not be construed as limiting the scope of the invention but as merely providing illustrations of some of the presently preferred embodiments of this invention. Various other embodiments and ramifications are possible within it's scope. For example, instead of the direct prostatic implants of androgen suppressive natural and synthetic steroidal and related chemical hormonal formulations, they may be implanted as subcutaneous or intramuscular implants for the treatment of prostate cancer especially as primary hormonal treatment of favorable prognostic early stage prostate cancer as alternative treatment by surgery or radiation therapy and for the treatment of hormone refractory advanced prostate cancer.

Thus the scope of the invention should be determined by the appended claims and their legal equivalents, rather than by the examples given.

What is claimed is:

1. An improved method of primary hormonal treatment with lesser or no toxic effect as primary treatment of early stage, low and intermediate risk prostate cancer and said primary hormonal treatment comprising of prostatic implants of steroid hormones, or its synthetic derivatives in one or more slow release formulations and permitting said drugs to be continuously released at near constant rate directly to prostate for longer periods and maintaining said formulation's serum level sufficient to effect suppression of androgen synthesis but low enough to minimize or to eliminate systemic toxicity.

2. A method according to claim 1, wherein said primary hormonal implant treatment of early stage, low and intermediate risk prostate cancer as an alternative to surgery and radiation therapy and the surgery or radiation therapy is reserved for those patients failing to said primary hormonal treatment

3. A method according to claim 1 further comprising release of said hormonal compositions to prostate for extended periods by diffusion and biodegradation from said prostatic implants in sufficient amounts to saturate the binding sites for said drug compositions in prostate and to exert their maximum tumor control activity and to follow up the biochemical tumor control by maintaining the serum PSA at a comparable low nadir value of 0.1 to 1 ng per ml as with post radiation therapy PSA values.

4. A method according to claim 1further comprising systemic maintenance of said drug compositions for extended periods by diffusion and biodegradation from said prostatic implants at an amount effective to suppress testicular and adrenal androgen synthesis with minimum or no systemic toxicity than if said drug compositions were administered orally or by intravenous, intramuscular or subcutaneous injections at much higher doses.

5. A method of claim 1 wherein said implants comprising of hormonally effective compositions selected from the natural or synthetic derivatives from the groups consisting of estrogens, progesterones, corticosteroids, from the anti-androgen groups consisting of flutamide, bicalutamide and nilutamide.

6. A method according to claim 1 wherein said prostatic implants of said drug compositions are made as separate or in combination thereof.

7. The method of claim 1 wherein said prostatic implants are made as biodegradable fused combinations of said therapeutic drug compositions and a lipoid carrier and said fused implants containing a single or multiples of said drug formulations for their slow release direct to prostate.

8. A method according to claim 1 wherein said prostatic implants are made of Silastic capsules containing said therapeutic drug compositions as separate or in combination thereof for said formulation's slow release direct to prostate.

9. The method of claim 1 wherein said prostatic implants are made as injectable microcapsules prepared from biodegradable polymer and said microcapsules containing said therapeutic drug compositions as separate or as in combination thereof for prostatic injection as slow release implant.

10. The method of claim 9, wherein said prostatic implants are made as injectable microcapsules prepared from biodegradable polymer and said microcapsules containing said therapeutic drug compositions dispensed in sterile liquid medium in sterile syringe for direct prostatic injection as slow release implant.

11. The method of claim 9, wherein said prostatic implants are made as injectable microcapsules prepared from biodegradable polymer and said microcapsules containing said therapeutic drug compositions dispensed in a mixture of sterile liquid mediums like normal saline, a local anesthetic and ethanol in a sterile syringe for direct prostatic injection as chelating slow release formulations when it comes in contact with tissue.

12. A method of claim 1, wherein implanting said implant compositions comprises of retropubic implants, trans perennial implant, trans rectal ultrasound based visualization of the prostate and implantation, computed tomography based visualization of prostate and implantation or by surgically exposing and free hand implanting.

13. The method of claim 1 wherein said prostatic implants are selected from readily available commercial pharmaceutical implant preparations of androgen suppressive steroid hormones or their derivatives and said implants containing a single or multiples of said drug formulations for their slow release direct to the prostate.

14. An improved method of concomitant hormonal and radiation treatment of prostate cancer and said hormonal treatment comprising of prostatic implants of steroid hormones in one or more slow release formulations and permitting said drugs to be continuously released at near constant rate directly to the prostate during the radiation therapy and afterwards for longer periods and maintaining said formulation's serum level sufficient to effect suppression of androgen synthesis but low enough to minimize or to eliminate their toxicity.

15. An improved method of concomitant hormonal and radiation treatment of prostate cancer according to claim 14, wherein said continued slow release of hormonal composition directly to the prostate during the interstitial radioactive seeds implants and afterwards for longer periods and maintaining said hormonal formulation's serum level sufficient to effect suppression of androgen synthesis but low enough to minimize or to eliminate their toxicity.

16. An improved method of concomitant hormonal and radiation treatment of prostate cancer according to claim 14, wherein said hormonal implants to prostate is performed concomitantly with the radioactive implants to improve cure and convenience to patient than when they are implanted separately.

17. A prostatic, subcutaneous or intramuscular implant method for hormonal treatment of prostate cancer for improved tumor control and less toxicity from hormonal treatment than by administering said hormonal compositions by oral, or intravenous routes and said hormonal treatment comprising of prostatic, subcutaneous or intramuscular implants of steroid hormones and or their synthetic derivatives in one or more slow release formulations.

18. A method of claim 17, wherein said prostatic, subcutaneous or intramuscular implants methods comprising single or synergetic combination of hormonally and cytotoxically effective compositions selected from natural or synthetic derivatives from the groups consisting of estrogens, progesterones, corticosteroids and from the anti-androgen groups consisting of flutamide, bicalutamide and nilutamide and they are fused with a lipoid carrier or encapsulated in Silastic capsules or formulated as injectable microcapsules as suitable slow-release prostatic, subcutaneous or intramuscular implant.

19. A method of claim 17, wherein said hormonally and cytotoxically effective compositions are continuously released at relatively constant rates to the systemic circulation by diffusion and biodegradation.

20. A method of claim 17, wherein said implants providing effective tumor control by suppression of hypothalamic LHRH and pituitary LH and FSH secretion and thereby suppression of testicular and adrenal androgen synthesis and or by their direct cytotoxic actions and said tumor control is evidenced by the decrease of serum PSA to a low nadir value of less than 1 ng per ml and serum acid phosphatase to less than 0.8 international unit, its upper limit of normal value.

21. A method of claim 17, wherein said slow-release subcutaneous or intramuscular implant for treating prostate cancer and providing minimum or no toxicity as compared to when said drug compositions were frequently administered orally or by intravenous injections at much higher doses to achieve the same rate of tumor control.

22. A method of claim 17, wherein when said implants are made as direct prostatic implants to reach said drug composition's high concentrations in the prostate and thereby to improve tumor control.

23. Slow-release anti-cancer prostate implants products for primary treatment of early stage T0-T2b prostate cancer before surgery or radiation therapy and comprising of a natural or synthetic estrogens and antiandrogens as fused with a lipoid carrier or as encapsulated in Silastic capsules or as injectable microcapsules and are suitable for prostatic implantation such that said hormonally and cytotoxically effective compositions are continuously released at relatively high constant rates to the prostate and their lower concentrations reaching the systemic circulation that is effective to suppress the testicular and adrenal androgen synthesis by inhibition of LHRH, FSH and LH secretions but with lesser toxicity.

24. The said products of claim 23 being further characterized by providing effective tumor control including biochemical tumor control evidenced by the decrease of PSA to a low nadir value of less than 1 ng per ml and acid phosphatase to less than 0.8 international unit, its upper limit of normal value and having minimum or no systemic toxicity associated with said composition's prostatic implants than if they were frequently administered orally or by intravenous, intramuscular or subcutaneous injections at much higher doses to achieve the same results.

25. Slow-release anti-cancer prostate implant product of claim 23, wherein said single drug formulation is made from any one of the drugs from a group consisting of DES, estradiol 17-β, iodoestradiol, progesterone, flutamide, bicalutamide, nilutamide and estramustine.

26. Slow-release anti-cancer prostate implant product of claim 23, wherein said synergetic two drugs formulations comprises of DES and prednisolone, DES and flutamide, DES and progesterone, estradiol 17-β and prednisolone, estradiol 17-β and progesterone, estradiol 17-β and flutamide, iodoestradiol and prednisolone, iodoestradiol and flutamide, iodoestradiol and progesterone, estramustine and prednisolone, estramustine and flutamide and estramustine and progesterone.

27. Slow-release anti-cancer prostate implant product of claim 23, wherein said synergetic three drugs formulations comprises of DES, prednisolone and flutamide, DES, flutamide and progesterone, estradiol 17-β, prednisolone and flutamide, estradiol 17-β, progesterone and flutamide, iodoestradiol, prednisolone and flutamide, iodoestradiol, progesterone and flutamide.

28. Anti-cancer products of claim 23, wherein said compositions comprising of single or synergetic combination of hormonally and cytotoxically effective amount of a natural or synthetic derivatives from the groups consisting of estrogens, progesterones, corticosteroids, from the anti-androgen groups consisting of flutamide, bicalutamide and nilutamide, and from the cytotoxic groups of drugs like estramustine in same or separate slow release biodegradable formulations as fused with a lipoid carrier suitable for prostatic implantation.

29. Anticancer products according to claim 23, wherein said single or synergetic combination of hormonally and cytotoxically effective amounts of formulations as fused with a lipoid carrier suitable for prostatic implantation such that said compositions are continuously released at relatively constant rates for longer periods and the contents of said compositions being kept in amounts effective to suppress tumor growth and to suppress testicular and adrenal androgen synthesis with minimum or no systemic toxicity than if said drug compositions were frequently administered orally or by intravenous, intramuscular or subcutaneous injections at much higher doses to achieve the same results as by said low dose prostatic implants.

30. Anticancer products of claim 23, wherein said single or synergetic combinations of hormonally and cytotoxically effective amounts of natural or synthetic estrogens, progesterones, cortisone and their derivatives, flutamide, bicalutamide and nilutamide, and estramustine in same or separate slow release Silastic capsules suitable for prostatic implantation.

31. Anticancer products according to claim 23 , wherein said single or synergetic combination of hormonally and cytotoxically effective amounts of formulations as slow release Silastic capsules suitable for prostatic implantation such that said compositions are continuously released at relatively constant rates for longer periods and the contents of said compositions being kept in amounts effective to suppress tumor growth and to suppress testicular and adrenal androgen synthesis with minimum or no systemic toxicity than if said drug compositions were frequently administered orally or by intravenous, intramuscular or subcutaneous injections at much higher doses to achieve the same results as by said low dose prostatic implants.

32. Anticancer products according to claim 23, wherein said anti-cancer products comprising of single or synergetic combination of hormonally and cytotoxically effective amounts of natural or synthetic derivatives from the groups consisting of estrogens, progesterones, corticosteroids, from the anti-androgen groups consisting of flutamide, bicalutamide and nilutamide in same or separate slow release injectable microcapsules suitable for prostatic implantation.

33. Anticancer products according to claim 32, wherein said single or synergetic combination of hormonally and cytotoxically effective amount of formulations as injectable microcapsules suitable for prostatic implantation such that said compositions are continuously released at relatively constant rates and the contents of said compositions being kept in amounts effective to suppress tumor growth and to suppress testicular and adrenal androgen synthesis with minimum or no systemic toxicity than if said drug compositions were frequently administered orally or by intravenous, intramuscular or subcutaneous injections at much higher doses to achieve the same results as by said low dose prostatic implants.

34. Anticancer products according to claim 23, wherein said implant products comprising of natural or synthetic derivatives from the groups consisting of estrogens, progesterones, corticosteroids, from the anti-androgen groups consisting of flutamide, bicalutamide and nilutamide and are made as separate or as mixtures of two or more thereof and fused with a lipid carrier.

35. Anti-cancer prostatic implant products according to claim 23, wherein said biodegradable prostatic implants comprise of natural or synthetic derivatives from the groups consisting of estrogens, progesterones, corticosteroids, from the anti-androgen groups consisting of flutamide, bicalutamide and nilutamide and are made as separate or as mixtures of two or more thereof as injectable microcapsules.

36. Anti-cancer prostatic implant products of claim 23, wherein said prostatic implants comprises of natural or synthetic derivatives from the groups consisting of estrogens, progesterones, corticosteroids, from the anti-androgen groups consisting of flutamide, bicalutamide and nilutamide and are made as separate or as mixtures of two or more thereof in Silastic capsules.

37. Anti-cancer prostatic implant products of claim 23, wherein said natural and synthetic estrogens, progesterones, cortisone and its derivative's serum level is being kept at low but sufficient concentration to suppress testicular and adrenal androgen synthesis and to minimize and or to eliminate systemic toxicity associated with them if they were administered orally or by intravenous, intramuscular or subcutaneous injections at much higher doses to effect androgen suppressive treatment of the prostate cancer.

38. A prostatic, subcutaneous or intramuscular slow-release hormonal implant method and products comprising single or synergetic combination of hormonally and cytotoxically effective compositions selected from the natural or synthetic derivatives from the groups consisting of estrogens, progesterones, corticosteroids, from the anti-androgen groups consisting of flutamide, bicalutamide and nilutamide and they are fused with a lipoid carrier or encapsulated in Silastic capsules or formulated as injectable microcapsules as suitable slow-release prostatic, subcutaneous or intramuscular implantation and implanting said products for the treatment of early and advanced stage prostate cancers or as hormonal treatment combined with radiation.

39. A method and product of claim 38, wherein said tumor control is evidenced by tumor regression and the return of pre-treatment elevated serum PSA to a low nadir value of less than 1 ng per ml and serum acid phosphatase to less than 0.8 international unit, its upper normal limit value.

40. A method and product of claim 38, wherein said hormone implant treatment as lesser-cost androgen suppressive treatment.

41. A method and product of claim 38, wherein said slow-release hormone implant treatment as hormonal prophylaxis against developing the prostate cancer in high risk group of men by slow release of androgen suppressive steroids from said hormone implants to the prostate in higher concentrations and their serum concentrations kept as low as just sufficient to suppress the androgen synthesis with none or minimal systemic toxicity and to follow up of any evidence of potential tumor development by periodic estimations of serum PSA and acid phosphatase which under said treatment will be at a low nadir value of less than 1 ng per ml for PSA and less than 0.8 international unit for serum acid phosphatase.
